(19) United States
(12) Patent Application Publication
(10) Pub. No.: US 2025/0009794 A1
(43) Pub. Date: Jan. 9, 2025

(54) PLACENTAL MACROPHAGES FOR CELL THERAPY

(71) Applicant: TECHNISCHE UNIVERSITÄT DRESDEN, Dresden (DE)

(72) Inventors: Michael SIEWEKE, Dresden (DE); Prashanth Kumar KANDALLA, Dresden (DE)

(21) Appl. No.: 18/713,492

(22) PCT Filed: Nov. 23, 2022
(86) PCT No.: PCT/EP2022/082951

§ 371 Date: May 24, 2024

(30) Foreign Application Priority Data
Nov. 26, 2021 (EP) 21 210 741.1-1118

(51) Int. Cl. A61K 35/15; C12N 5/00; C12N 5/0786

(52) U.S. Cl. CPC A61K 35/15; C12N 5/0087; C12N 5/0645; C12N 2509/00

(57) ABSTRACT

The present invention to the use of human term placenta as a source of HLA-matched cells for use in cell therapy, in particular wherein the cells are non-maternal macrophages. The invention also relates to two different sets of isolated placental macrophages of embryonic origin, which have been newly discovered and identified as being useful for macrophage cell therapy, and to methods to isolate these cells from human term placenta.

### DEFINITIONS

The term “gene” means a DNA sequence that codes for an RNA or a particular sequence of amino acids which comprise all or part of one or more proteins or enzymes, and may or may not include regulatory DNA sequences, such as promoter sequences, which determine for example the conditions under which the gene is expressed. A “promoter” or “promoter sequence” is a DNA regulatory region capable of binding RNA polymerase in a cell and initiating transcription of a downstream (3′ direction) coding sequence. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

Within the context of the present invention the terms “mutant” and “mutation” mean a detectable change in genetic material, i.e. genomic DNA. Mutations include deletion, insertion or substitution of one or more nucleotides. The mutation may occur in the coding region of a gene (i.e. in exons), in introns, or in the regulatory regions (e.g. enhancers, response elements, suppressors, signal sequences, polyadenylation sequences, promoters) of the gene. Generally, a mutation is identified in a subject by comparing the sequence of a nucleic acid or polypeptide expressed by said subject with the corresponding nucleic acid or polypeptide expressed in a control population. Where the mutation is within the gene coding sequence, the mutation may be a “missense” mutation, where it replaces one amino acid with another in the gene product, or a “nonsense” mutation, where it replaces an amino acid codon with a stop codon. A mutation may also occur in a splicing site where it creates or destroys signals for exon-intron splicing and thereby lead to a gene product of altered structure. Within the context of the present invention a mutation is not silent, i.e. it results at least in an alteration of the nucleotide sequence (where the gene product is a functional RNA) or an amino acid sequence (where the gene product is a protein) that renders the gene product non-functional or that reduces expression of the gene product at the RNA-level by at least 80%. Preferred mutations, for example deletions of whole genes, abolish gene expression.

As used herein the term “deletion” means that a part of a DNA-sequence is missing compared to a reference sequence.

As used herein gene expression is “inhibited” when expression of the gene at the RNA-level is reduced by at least 80% compared to gene expression in the corresponding wildtype, as measured by quantitative rt-PCR. Preferably expression of the gene at the RNA-level is reduced by at least 90%, such as by at least 95%.

As used herein gene expression is “abolished” when expression of the gene is not detectable at the RNA-level by q-PCR. In a qPCR an mRNA which is “expressed” and thus present at detectable levels will a) give a sigmoidal fluorescence curve that b) reaches a plateau at least within 38 PCR-cycles, preferably at least within 36 PCR-cycles, and c) produces a PCR-product of the expected length, i.e. corresponding in length to a PCR-product derived from mature mRNA and not from genomic DNA or unprocessed RNA intermediates. Preferably mRNA expression can be confirmed by these three criteria in three repeated qPCR experiments.

“Mutagenesis” as used herein is a laboratory process by which the genetic information of an organism is deliberately changed, resulting in a mutation. Preferred methods for mutagenesis herein are methods based on site-specific endonucleases.

A “site specific endonuclease” as used herein is an enzyme that cleaves a phosphodiester bond within a polynucleotide chain only at a very specific nucleotide sequence in the middle (endo) portion of a double-stranded DNA molecule, which sequence occurs preferably only once within the whole human genome so as to allow specific genetic engineering of a human target cell. Examples of site-specific endonucleases, which are typically used for genetic engineering in human target cells are zinc finger nucleases, TALENs and the CRISPR/Cas9 system.

As used herein the term “expression” may refer to gene expression of a polypeptide or protein, or to gene expression of a polynucleotide, such as miRNAs or lncRNAs, depending on the context. Expression of a polynucleotide may be determined, for example, by measuring the production of RNA transcript levels using methods well known to those skilled in the art. Expression of a protein or polypeptide may be determined, for example, by immunoassay using (an) antibody(ies) that binds the polypeptide specifically, using methods well known to those skilled in the art.

As used herein the “expression of an mRNA” relates to the transcriptional level of gene expression.

In the present invention, known methods can be used to detect such an expression of a gene. Examples of the method for quantitatively detecting an mRNA level in a cell or collection of cells include for example PCR-based methods (real-time PCR, quantitative PCR), and DNA microarray analysis. In addition, an mRNA level can be quantitatively detected by counting the number of reads according to what is called a new generation sequencing method. Exemplary methods, conditions and materials to be used for the determination of the expression level of an mRNA are described in the experimental section of this disclosure. A preferred method for determining expression of an mRNA is qPCR, as explained under “abolished expression” above.

Those skilled in the art can prepare an mRNA or a nucleic acid cDNA to be detected by the aforementioned detection methods by taking the type and state of the specimen and so forth into consideration and selecting a known method appropriate therefor. When the gene expression level in human macrophages of the invention is compared with the expression level of the same gene in wildtype macrophages, it is compared under otherwise identical conditions, i.e. both types of macrophages are to be cultured and treated in the same manner in order to allow for a scientifically meaningful comparison of mRNA levels.

As used herein, “very high” expression of an mRNA is defined as at least 10% of the gene expression level of the household gene GAPDH as measured at the mRNA level. GAPDH is the gene encoding glyceraldehyde-3-phosphate dehydrogenase, described in detail as gene ID 2597 in the NCBI Gene database.

As used herein, “high” expression of an mRNA is defined as from 1% to 10% of the gene expression level of the household gene GAPDH, “mediocre” expression of an mRNA is defined as from 0.1% to 1% of the gene expression level of the household gene GAPDH and “low” is defined as from 0.005% to 0.1% of the gene expression level of the household gene GAPDH.

An “allele” as used herein refers to one of the two copies of the same human gene. The two copies of a gene, one each on the two homologous chromosomes, can be identical or vary slightly in their individual sequences. The term allele is thus used slightly differently from its typical use herein, because it includes identical versions of the same human gene on the same relative place on the two homologous chromosomes. In a diploid cell, the two alleles of a given gene occupy corresponding loci on a pair of homologous chromosomes.

As used herein an “allele” or a “gene” is rendered nonfunctional if no further expression of the protein encoded by the gene or allele is detectable after the procedure that rendered the allele or gene nonfunctional. That is, no new protein is being expressed from an allele or gene that has been rendered nonfunctional. Eventually the protein encoded by an allele or gene that has been rendered nonfunctional will become undetectable in a population of cells consisting of cells with only nonfunctional alleles. The time until the protein encoded by said gene or allele will become undetectable depends on the dynamics of protein and mRNA turnover for said gene.

As used herein the term “deletion” means that a part of a DNA-sequence is missing compared to a wildtype reference sequence.

As used herein an “exon” is any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

The term “guide RNA” as used herein relates to “guide RNA” as used in the context of a CRISPR/Cas9 DNA editing system. The guide RNA confers target sequence specificity to the CRISPR-Cas9 system. Guide RNAs are non-coding short RNA sequences which first bind to the Cas9 enzyme and then the guide RNA sequence guides the complex via base pairing to a specific location on the DNA, where Cas9 acts as an endonuclease and cuts the target DNA strand. Examples of guide RNAs are a) a synthetic trans-activating CRISPR RNA (tracrRNA) plus a synthetic CRISPR RNA (crRNA), wherein the crRNA is designed to identify the gene target site of interest, and b) a single guide RNA (sgRNA) that combines both the crRNA and tracrRNA within a single construct.

The term “MAF” denotes the human MAF transcription factor. MAF and other Maf family members form homodimers and heterodimers with each other and with Fos and Jun, consistent with the known ability of the AP-1 proteins to pair with each other (Kerppola and Curran (1994); Kataoka, K. et al. (1994)). The gene for human MAF is located on chromosome 16, location 16q23.2 and is described in detail as Gene ID 4094 in the NCBI Gene database. Sequence and location information refer to the annotation release 109.20201120, which is the current release on Dec. 9, 2020, Reference sequence assembly GCF_000001405.39 of the Genome Reference Consortium Human Build 38 patch release 13. This reference identifies the gene for MAF on the complementary strand between 79,593,838 and 79,600,737.

The term “MAFB” denotes the human MAFB transcription factor. This gene is expressed in a variety of cell types (including lens epithelial, pancreas endocrine, epidermis, chondrocyte, neuronal and hematopoictic cells, in particular macrophages) and encodes a protein containing a typical bZip motif in its carboxy-terminal region. In the bZip domain, MAFB shares extensive homology not only with MAF but also with other Maf-related proteins. MAFB can form a homodimer through its leucine repeat structure and specifically binds Maf-recognition elements (MAREs) palindromes, composite AP-1/MARE sites or MARE halfsites with AT rich 5′ extensions (Yoshida, et al. 2005). In addition, MAFB can form heterodimers with Maf or Fos through its zipper structure but not with Jun or other Maf family members (Kataoka et al., 1994). The gene for human MAFB is located on chromosome 20, location 20q12 and is described in detail as Gene ID 9935 in the NCBI Gene database. Sequence and location information refer to the annotation release 109.20201120, which is the current release on Dec. 9, 2020, Reference sequence assembly GCF_000001405.39 of the Genome Reference Consortium Human Build 38 patch release 13. This reference identifies the gene for MAFB on the complementary strand between 40685848 and 40689236.

HLA-A or “human leukocyte antigen 1” relates to a protein belonging to the HLA class I heavy chain paralogues. This class I molecule is a heterodimer consisting of a heavy chain and a light chain (beta-2 microglobulin). The heavy chain is anchored in the membrane. Class I molecules play a central role in the immune system by presenting cytosolic peptides shuttled to the endoplasmic reticulum lumen so that they can be recognized by cytotoxic T cells. They are expressed in nearly all cells. The heavy chain is approximately 45 kDa and its gene contains 8 exons. Exon 1 encodes the leader peptide, exons 2 and 3 encode the alpha1 and alpha2 domains, which both bind the peptide, exon 4 encodes the alpha3 domain, exon 5 encodes the transmembrane region, and exons 6 and 7 encode the cytoplasmic tail. Polymorphisms within exon 2 and exon 3 are responsible for the peptide binding specificity of each class one molecule. Typing for these polymorphisms is routinely done for bone marrow and kidney transplantation. More than 6000 HLA-A alleles have been described. The gene for human HLA-A is located on chromosome 6, location 6p22.1 and is described in detail as Genc ID 3105 in the NCBI Gene database. Sequence and location information refer to the annotation release 109.20201120, which is the current release on Dec. 9, 2020, Reference sequence assembly GCF_000001405.39 of the Genome Reference Consortium Human Build 38 patch release 13. This reference identifies the gene for HLA-A on the coding strand between 29942532 and 29945870.

HLA-B relates to a protein belonging to the HLA class I heavy chain paralogues. Hundreds of HLA-B alleles have been described. The gene for human HLA-B is located on chromosome 6, location 6p21.33 and is described in detail as Gene ID 3106 in the NCBI Gene database. Sequence and location information refer to the annotation release 109.20201120, which is the current release on Dec. 9, 2020, Reference sequence assembly GCF_000001405.39 of the Genome Reference Consortium Human Build 38 patch release 13.

HLA-DR is a dimeric protein belonging to the HLA class II. The HLA class II molecule is a heterodimer consisting of an alpha (such as HLA-DRA) and a beta chain (such as HLA-DRB1, HLA-DRB3, HLA-DRB4 or HLA-DRB5), both anchored in the membrane. It plays a central role in the immune system by presenting peptides derived from extracellular proteins. Class II molecules are expressed in antigen presenting cells. As the alpha chain is practically invariable, variability in the composition of HLA-DR within an individual derives mostly from the ability of the alpha chain to pair with the beta chain from three different DR beta loci, HLA-DRB1 and two of any DRB3, DRB4 or DRB5 alleles. Within the DR molecule the beta chain contains essentially all the polymorphisms specifying the peptide binding specificities. In particular hundreds of DRB 1 alleles have been described and some alleles have increased frequencies associated with certain diseases or conditions. These alleles are responsible for variability in the composition of HLA-DR within a population, and an individual can be homozygous with regard to HLA-DRB1 (i.e. father and mother carried the same allele of HLA-DRB1), but in general most individuals are heterozygous for HLA-DRB1. The gene for human HLA-DRB1 is located on chromosome 6, location 6p21.32 and is described in detail as Gene ID 31223 in the NCBI Gene database. Sequence and location information refer to the annotation release 109.20201120, which is the current release on Dec. 9, 2020, Reference sequence assembly GCF_000001405.39 of the Genome Reference Consortium Human Build 38 patch release 13.

“Blood type antigens A and B” as used herein relate to the two antigens of the ABO-blood group system. The two antigens are antigen A and antigen B and are present on the red blood cells. Regarding the antigen property of the blood all human beings can be classified into 4 groups, those with antigen A (group A), those with antigen B (group B), those with both antigen A and B (group AB) and those with neither antigen (group O). Preferred placentas are derived from embryos which do not have the blood-type antigens A and B and are thus of blood group O.

“Rhesus D antigen” as used herein relates to antigen D of the Rh-blood group system. The Rh blood group system consists of 49 defined blood group antigens, among which the five antigens D, C, c, E, and e are the more important, with antigen D being defining for being Rh positive or Rh negative. Somebody who is Rh positive has the Rh D antigen, and somebody without the Rh D antigen is negative. Preferred placentas are derived from embryos which do not have the Rhesus D antigen and are thus Rh negative.

The nomenclature chosen herein for the description of HLA alleles and haplotypes follows the one used by Taylor et al. (2012) in Cell Stem Cell.

LYVE1 as used herein relates, depending on the context, to the LYVE1 gene or to a surface marker, which is the extracellular part of the LYVE1 protein, the lymphatic vessel hyaluronan receptor 1. LYVE1 is described in detail as gene ID 10894 in the NCBI Gene database.

IL18RAP as used herein relates, depending on the context, to the IL18RAP gene or to the surface marker CDw218b which is the extracellular part of the IL18RAPgene protein, the interleukin 18 receptor accessory protein. IL18RAP is described in detail as gene ID 8807 in the NCBI Gene database.

PPBP as used herein relates, depending on the context, to the PPBP gene or to the secreted protein CXCL7, which is a growth factor that belongs to the CXC chemokine family and a potent chemoattractant and activator of neutrophils. PPBP is described in detail as gene ID 5473 in the NCBI Gene database.

S100A12 as used herein relates, depending on the context, to the S100A12 gene or to the extracellular calcium binding protein A12. S100A12 is described in detail as gene ID 6283 in the NCBI Gene database.

SERPINB2 as used herein relates, depending on the context, to the SERPINB2 gene or to the extracellular protein plasminogen activator inhibitor-2, which is a serine protease inhibitor of the serpin superfamily that inactivates tissue plasminogen activator and urokinase. SERPINB2 is described in detail as gene ID 5055 in the NCBI Gene database.

PTGES as used herein relates, depending on the context, to the PTGES gene or to the enzyme prostaglandin synthase. PTGES is described in detail as gene ID 9536 in the NCBI Gene database.

LRG1 as used herein relates, depending on the context, to the LRG1 gene or to the secreted form of the leucine rich alpha-2-glycoprotein. LRG1 is described in detail as gene ID 116844 in the NCBI Gene database.

CFP as used herein relates, depending on the context, to the CFP gene or to the secreted form of CFP which is plasma glycoprotein that positively regulates the alternative complement pathway of the innate immune system. CFP protein binds to many microbial surfaces and apoptotic cells and stabilizes the C3- and C5-convertase enzyme complexes in a feedback loop that ultimately leads to formation of the membrane attack complex and lysis of the target cell. CFP is described in detail as gene ID 5199 in the NCBI Gene database.

PTX3 as used herein relates, depending on the context, to the PTX3 gene or to pentraxin 3, a secreted protein which promotes fibrocyte differentiation and is involved in regulating inflammation and complement activation. PTX3 is described in detail as gene ID 5806 in the NCBI Gene database.

APOBEC3A as used herein relates, depending on the context, to the APOBEC3A gene or to the enzyme, which is a single-domain DNA cytidine deaminase with antiviral effects. APOBEC3A is described in detail as gene ID 200315 in the NCBI Gene database.

CD55 as used herein relates, depending on the context, to the CD55 gene or to a surface marker, which is the extracellular part of the CD55 protein, a glycoprotein involved in the regulation of the complement cascade. CD55 is described in detail as gene ID 1604 in the NCBI Gene database.

MRC1 as used herein relates, depending on the context, to the MRC1 gene or to the surface marker CD206, which is the extracellular part of the mannose receptor C-type 1, a type I membrane receptor that mediates the endocytosis of glycoproteins by macrophages. MRC1 is described in detail as gene ID 4360 in the NCBI Gene database.

FCAR as used herein relates, depending on the context, to the FCAR gene or to the surface marker CD89, which is the extracellular part of the myeloid receptor for the Fc region of IgA, a transmembrane glycoprotein present on the surface of myeloid lineage cells such as neutrophils, monocytes, macrophages, and cosinophils, where it mediates immunologic responses to pathogens. FCAR is described in detail as gene ID 2204 in the NCBI Gene database.

PLTP as used herein relates, depending on the context, to the PLTP gene or to the phospholipid transfer protein, a secreted protein which mediates the transfer of phospholipids and free cholesterol from triglyceride-rich lipoproteins (low density lipoproteins or LDL and very low density lipoproteins or VLDL) into high-density lipoproteins (HDL) as well as the exchange of phospholipids between triglyceride-rich lipoproteins themselves. PLTP is described in detail as gene ID 5360 in the NCBI Gene database.

CCL18 as used herein relates, depending on the context, to the CCL18 gene or to the secreted chemokine CCL18, which is a potent chemoattractant for naïve T-cells. CCL18 is described in detail as gene ID 6362 in the NCBI Gene database.

RNASE1 as used herein relates, depending on the context, to the RNASE1 gene or to the secreted ribonuclease RNASE1, which is an endonuclease degrading extracellular RNA. RNASE1 is described in detail as gene ID 6035 in the NCBI Gene database.

LGMN as used herein relates, depending on the context, to the LGMN gene or to the cysteine protease legumain, which is thought to be involved in the processing of bacterial peptides and endogenous proteins for MHC class II presentation. LGMN is described in detail as gene ID 5641 in the NCBI Gene database.

C1QB as used herein relates, depending on the context, to the C1QB gene or to the secreted complement C1q B chain, which is the B-chain polypeptide of serum complement subcomponent C1q, which associates with C1r and C1s to yield the first component of the serum complement system. C1QB is described in detail as gene ID 713 in the NCBI Gene database.

ADORA3 as used herein relates, depending on the context, to the ADORA3 gene or to the membrane protein adenosine A3 receptor, which is a G-protein coupled receptor for adenosine. It is involved in the inhibition of neutrophil degranulation in neutrophil-mediated tissue injury. ADORA3 is described in detail as gene ID 140 in the NCBI Gene database.

SIGLEC11 as used herein relates, depending on the context, to the SIGLEC11 gene or to a surface marker, which is the extracellular part of the SIGLEC11 protein, a cell surface lectin of the sialic acid-binding immunoglobulin-like lectin family. SIGLEC11 mediates anti-inflammatory and immunosuppressive signaling and is described in detail as gene ID 114132 in the NCBI Gene database.

The term “proliferating cell” as used herein refers to a cell that is capable of cell division. A cell is a proliferating cell if a population of at least 1000 “proliferating cells” increases in cell number by at least 4-fold after 8 days under suitable cultivation conditions, i.e. when n (192 h)/n (0 h) is at least 4,00 with n being the total number of cells in the cell population at the indicated time points.

The term “differentiated human cell” as used herein is a cell that does not change cell type and even upon cell division gives rise to two cells of the same cell type. This is in contrast to “pluripotent cells” which can differentiate into all cell types of the adult organism and oligopotent cells, which can differentiate into a few closely related cell types.

A “myeloid cell” as used herein is a cell of hematopoietic origin that is not lymphoid and not erythro-megakaryocytic and not a multi-lineage progenitor with more than myeloid lineage potential.

A “monocyte” is a mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kindey-shaped). It may fold over on top of itself, thus showing brainlike convolutions. No nucleoli are visible. The chromatin pattern is fine, and arranged in skein-like strands. The cytoplasm is abundant and appears blue gray with many fine azurophilic granules, giving a ground glass appearance in Giemsa staining. Vacuoles may be present. More preferably, the expression of specific surface antigens is used to determine whether a cell is a monocyte cell. Phenotypic markers of human monocyte cells include CD11b, CD11c, CD33, CD45 and CD115. Generally, human monocyte cells express CD9, CD11b, CD11c, CDw12, CD13, CD15, CDw17, CD31, CD32, CD33, CD35, CD36, CD38, CD43, CD45, CD49b, CD49c, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDw121b, CDw123, CD127, CDw128, CDw131, CD147, CD155, CD156a, CD157, CD162, CD163, CD164, CD168, CD171, CD172a, CD180, CD131a1, CD213a2, CDw210, CD226, CD281, CD282, CD284, CD286 and optionally CD4, CD14, CD16, CD40, CD45RO, CD45RA, CD45RB, CD62L, CD74, CD141, CD142, CD169, CD170, CD181, CD182, CD184, CD191, CD192, CD194, CD195, CD197, CD206, CX3CR1.

A “macrophage” is a myeloid cell of the innate immune system exhibiting properties of phagocytosis. The morphology of macrophages varies among different tissues and between normal and pathologic states, and not all macrophages can be identified by morphology alone. However, most macrophages are large cells with a round or indented nucleus, a well-developed Golgi apparatus, abundant endocytotic vacuoles, lysosomes, and phagolysosomes, and a plasma membrane covered with ruffles or microvilli. The key functions of macrophages in innate and adaptive immunity are the phagocytosis and subsequent degradation of senescent or apoptotic cells, microbes and neoplastic cells, the secretion of cytokines, chemokines and other soluble mediators, and the presentation of foreign antigens (peptides) on their surface to T lymphocytes. Macrophages are derived from multipotent progenitor cells, common myeloid progenitor cells and granulocyte-monocyte progenitor cells in the bone marrow of mammalian organisms, which ultimately develop through further progenitor stages into monocytes that then enter the peripheral bloodstream. Unlike neutrophils, with their multilobed nuclei, monocytes have kidney-shaped nuclei and assume a large cell body during further differentiation and activation. Throughout life, some monocytes adhere to and migrate through the endothelium of the capillaries into all organs, where some of them can differentiate into resident tissue macrophages or dendritic cells (see below). Besides monocyte origin, tissue resident macrophages can also develop from early primitive macrophage progenitors of the yolk sac from before the establishment of definitive hematopoiesis, from erythroid-macrophage progenitors (EMP) of diverse hematopoietic sites of the embryo or from embryonic hematopoietic stem cell derived fetal monocytes. These embryo derived macrophages can persist into adulthood and be maintained long term independently of input from adult hematopoietic stem cells and monocytes. Lymphatic tissues, such as the lymph nodes and the spleen, are particularly rich in macrophages but tissue resident macrophages are present in essentially every organ of the body.

In the context of the invention, the macrophages are placental macrophages.

Macrophages are an important source of cytokines. Functionally, the numerous cytokine products can be placed into several groups: (1) cytokines that mediate a proinflammatory response, i.e. help to recruit further inflammatory cells (e.g. IL-1, II-6, TNFs, CC and CXC chemokines, such as IL-8 and monocyte-chemotactic protein 1); (2) cytokines that mediate T cell and natural killer (NK) cell activation (e.g. IL-1, IL-12, IL-15, IL-18); (3) cytokines that exert a feedback effect on the macrophage itself (e.g. IL-1, TNFs, IL-12, IL-18, M-CSF, IFNα/β, IFNγ); (4) cytokines that downregulate the macrophage and/or help to terminate the inflammation (e.g. IL-10, TGFβs), (5) cytokines important for wound healing or to support tissue stem cells (e.g. EGF, PDGF, bFGF, TGFβ) or to support blood vessel growth (e.g. VEGF) or neurons (e.g. neurotrophic factors, kinins). The production of cytokines by macrophages can be triggered by microbial products such as LPS, by interaction with type 1 T-helper cells, or by soluble factors including prostaglandins, leukotrienes and, most importantly, other cytokines (e.g. IFNγ). Generally, human macrophages express CD11c, CD11b, CD14, CD18, CD26, CD31, CD32, CD36, CD45RO, CD45RB, CD63, CD68, CD71, CD74, CD87, CD88, CD101, CD115, CD119, CD121b, CD155, CD156a, CD204, CD206 CDw210, CD281, CD282, CD284, CD286 and in a subset-specifc manner CD163, CD169 CD170, MARCO, FOLR2, LYVE1. Activated macrophages can further express CD23, CD25, CD69, CD105 and HLA-DR, HLA-DP and HLA-DQ

The terms “phagocytic cells” and “phagocytes” are used interchangeably herein to refer to a cell that is capable of phagocytosis. There are different main categories of professional phagocytes: mononuclear phagocytes, comprising macrophages sensu strictu, monocytes and dendritic cells as well as polymorphonuclear leukocytes (neutrophils). However, there are also “non-professional” phagocytic cells known to participate in efferocytosis, efferocytosis being the process by which professional and nonprofessional phagocytes dispose of apoptotic cells in a rapid and efficient manner.

The term “progenitor cell” as used herein relates to cells which are descendants of stem cells and which can further differentiate to create specialized cell types. There are many types of progenitor cells throughout the human body. Each progenitor cell is only capable of differentiating into cells that belong to the same tissue or organ. Some progenitor cells have one final target cell that they differentiate to, while others have the potential to terminate in more than one cell type. Progenitor cells are thus an intermediary cell type involved in the creation of mature cells in human tissues and organs, the blood, and the central nervous system. Hematopoietic progenitor cells are an intermediate cell type in blood cell development. They are immature cells that develop from hematopoietic stem cells and eventually differentiate into one of more than ten different types of mature blood cells.

The term “CD34+ multipotent progenitors” as used herein are a CD34 surface antigen expressing stem-cell enriched hematopoietic progenitor population, that are not macrophages, monocytes or dendritic cells.

A “surface marker” is a molecule, typically a protein or a carbohydrate structure, that is present and accessible on the exterior of the plasma membrane of a cell and that is specific for a particular cell type or a limited number of cell types, thereby being a “marker” for these cell types. Examples of surface markers on human macrophages are CD11c, CD11b, CD14, CD16, CD18, CD26, CD31, CD32, CD33, CD36, CD45RO, CD45RB, CD63, CD64, CD68, CD71, CD74, CD87, CD88, CD101, CD115, CD119, CD121b, CD155, CD156a, CD163, CD169, CD170, CD204, CD206 CDw210, CD281, CD282, CD284, CD286, MARCO, FOLR2, CX3CR1 and LYVE1.

A cell is “positive” for a surface marker if staining with a surface-marker-specific antibody creates a specific fluorescence signal in a FACS experiment. The principles of FACS are explained in detail in the book “practical flow cytometry”, 4th edition by Howard M. Shapiro. In a FACS experiment a collection of cells is typically stained with several fluorescent antibodies, each one selectively binding a different surface marker and having a different fluorochrome. This allows the selection of particular cell types within a heterogeneous collection of cells by appropriate gating strategies in a FACS experiment. A specific fluorescence signal by the surface-marker-specific antibody is typically then verified in a one-dimensional histogram plot by comparing the histograms for the staining with all antibodies with the histogram for the staining with the mix of antibodies where only the surface-marker-specific antibody has been omitted (so called “FMO” or “fluorescence minus one” signal). If the two histograms are different such that the staining with the mix of all antibodies produces more fluorescence than the FMO control, then the tested collection of cells is positive for the tested cell surface marker. In terms of visual appearance of the histogram this means that the peak of fluorescence for the staining with the mix of all antibodies is shifted to higher fluorescence values when compared to the FMO control. Preferably the two histograms-all antibodies on the one hand and FMO on the other-overlap by at most 70 area % (area under the curve), such as at most 50 area %, for example by at most 25 area %.

The term “collection of cells” as used herein relates to at least 10000 cells, which cells are alive.

The term “expansion” of cells as used herein is the process of culturing cells under suitable laboratory conditions and increasing the number of living cells by mitotic divisions of the cultured cells.

The term “genetically modified” cell as used herein relates to a cell wherein the cell's DNA has been changed using biotechnological methods. For example, cells wherein the cells' DNA has been manipulated by the use of a CRISPR/Cas9 DNA editing system, wherein the manipulation has left a detectable change in the cells' DNA, are genetically modified cells.

The term “ex-vivo” as used herein means outside of a living body.

The term “in-vitro” as used herein means outside of a living body and within a laboratory environment. For example, cells which are cultured “in-vitro” are cultured in controlled, and often artificial, culture media.

“Placenta” as used herein relates to the temporary fetal organ that facilitates nutrient, gas and waste exchange between the mother and the fetus. As used herein “placenta” does not include the umbilical cord, the chorion and the amnion and is preferably term placenta, which is obtainable as the afterbirth after child delivery or, preferably, following birth by a cesarian section.

A “placenta macrophage” as used herein is a mononuclear phagocytic cell having macrophage-specific surface markers, which macrophage is obtainable from placenta. A “placenta macrophage” as used herein is of embryonic origin and does not originate from the mother. Maternal macrophages are also present in the placenta, but those maternal macrophages are not comprised in the term “placenta macrophage” as used herein. A fetal placenta-resident monocyte which is not a cord blood monocyte can be considered to be a placental macrophage. Placental macrophages are different from macrophages of adult human beings, for example in that they show typical features of very young cells. For example, the skilled person may identify fetal macrophages because their transcriptomic signatures are more related to published transcriptomes of fetal macrophages than to published transcriptomes of macrophages from adult human beings. For example, the skilled person may identify fetal macrophages because their epigenetic signatures of histone or DNA modifications are more related to published epigenomes of fetal and newborn macrophages than to published epigenomes of macrophages from adult human beings. For example, the skilled person may identify fetal macrophages because their telomeres are longer than those of macrophages from adult human beings. For example, the skilled person may identify placental macrophages based on having lower levels of metabolic senescence markers, such as, for example, lipofuscin levels, than macrophages from adult human beings. For example, the skilled person may identify placental macrophages based on smaller cell size, and/or increased mitochondrial quality and activity and/or increased autophagic capacity compared to adult macrophages. For example, the skilled person may identify placental macrophages based on them having higher levels of metabolic markers of young cells, such as, for example, catalase activity and/or GSH levels, than macrophages from adult human beings.

By “purified” and “isolated” it is meant, when referring to a cell or a population of cells, that said cell or said population of cells is present in the substantial absence of other cells or population of cells. The term “purified” as used herein preferably means at least 75% by number, more preferably at least 85% by number, still preferably at least 95% by number, and most preferably at least 98% by number, of cells of the same type are present.

As used herein, the term “subject” denotes a human being.

In the context of the invention, the term “treating” or “treatment”, as used herein, means reversing, alleviating, inhibiting the progress of, or preventing the disease or condition to which such term applies, or one or more symptoms of such disease or condition. Preferably it means reversing, alleviating, inhibiting the progress of, or preventing the disease or condition.

As used herein “autologous” is a term referring to an individual's own cells. For example, in autologous blood transfusions, the patient's own blood is collected and reinfused into the body.

As used herein “allogeneic” is a term referring to human cells that are not an individual's own cells. For example, an allogeneic stem cell transplant is different from an autologous stem cell transplant, which uses stem cells from the patient's own body.

As used herein, “regenerative medicine” relates to the use of cells, such as macrophages, in restoring the functionality of tissues that have been injured by trauma, damaged by disease or worn by time. An example for a use of macrophages in the context of regenerative medicine is their use in wound healing.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

It is to be understood that this invention is not limited to the particular materials and methods described herein. It is also to be understood that the terminology used herein is for the purpose of describing particular embodiments and is not intended to limit the scope of the present invention, which will be limited only by the appended claims. As used herein, the singular forms “a”, “an”, and “the” include plural reference unless the context clearly indicates otherwise.

Unless defined otherwise, all technical and scientific terms used herein have the same meanings as commonly understood by one of ordinary skill in the art to which this invention belongs. The following references provide one of skill with a general definition of many of the terms used in this invention-unless defined otherwise herein—and ranked in increasing order of priority: Singleton et al., Dictionary of Microbiology and Molecular Biology (3rd ed. 2006); The Glossary of Genomics Terms (JAMA. 2013; 309 (14): 1533-1535), Janeway's Immunobiology, 9th edition and “Practical Flow Cytometry”, 4th edition by H.M. Shapiro.

All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### DETAILED DESCRIPTION

The present invention relates to the use of human term placenta as a starting material for the preparation of embryo-derived somatic cells for use as a medicament. The present invention shows that embryo-derived cells from human term placenta are functional in cell therapy, thus rendering human term placenta useful as a source of somatic, embryo-derived cells. Somatic cells from human term placenta can be fetal syncytiotrophoblasts, fetal mesenchymal cells, fetal endothelial cells or fetal vascular or perivascular cells. Preferred embryo-derived cells from human term placenta are, however, embryo-derived somatic mononuclear phagocytic cells, and in particular embryo-derived human placental macrophages.

Fetal cells are present in the blood of a pregnant woman (Herzenberg et al. (1979) PNAS 76 (3): 1453-1455). In particular fetal nucleated red blood cells or circulating trophoblasts are presently used for noninvasive prenatal diagnostic methods and even for whole genome profiling. In particular circulating trophoblasts can be isolated based on expression of specific biomarker signatures and then analyzed by techniques like whole genome amplification and next generation sequencing. Thus, the sequence of specific regions of the embryo's genome, such as the embryo's HLA-genes, can be determined (Breman et al. (2016) Prenat. Diagn. 36 (11): 1009-1019; see also Pin-Jung et al. (2019) Curr. Obstet. Gynecol. Rep. 8 (1): 1-8 for a review).

Since therefore the haplotype of an embryo with regard to the HLA-genes can be determined during pregnancy, a suitable, and preferably haploidentical, recipient for the embryo-derived somatic cells from human term placenta can also be identified already prior to birth. The invention therefore also relates to the use of human term placenta as a source of haplotype-matched placental cells, and in particular embryo-derived somatic mononuclear phagocytic cells, such as embryo-derived human placental macrophages, for use as a medicament, e.g. as a cell therapy.

When the immune haplotype of an embryo is determined prior to birth, certain features determine whether the embryo-derived somatic cells from human term placenta will be compatible with more or fewer potential recipients. A preferred feature is that the embryo should be of blood type 0. A preferred feature is that the embryo should be negative for the Rhesus D-Antigen.

A preferred feature is that the embryo should be homozygous for one, two or all of the genes encoding the human leukocyte antigens HLA-A, HLA-B and HLA-DR. The combination of HLA-A, HLA-B and HLA-DR genes is not random because of genetic recombination and external pressures from environmental factors, resulting in linkage disequilibrium. According to Taylor et al. (2012), referring to the 2010 WHO HLA nomenclature report, there are 21 HLA-A, 44 HLA-B and 15 HLA-DR split specificities, generating a total of 13860 HLA combinations. These HLA-combinations are not equally distributed among the population of any particular country, and some HLA-combinations of homozygous donors produce an HLA-match with more potential recipients within a population as others. For example, the two homozygous HLA-combinations for which the most potential recipients are found within the population of the UK are HLA-A1, HLA-B8, HLA-DR 17 (3) and HLA-A2, HLA-B44 (12), HLA-DR4. Such embryo-derived placental cells are therefore particularly useful, wherein the embryo is homozygous for HLA-A, HLA-B and HLA-DR and wherein the HLA-A, HLA-B and HLA-DR combination is one of the 10 most frequent combinations within a population selected from the group consisting of inhabitants of the European Union, inhabitants of the United States of America, inhabitants of the China and inhabitants of Japan.

In practice, the fetal haplotype with regard to HLA-A, HLA-B and HLA-DR will be determined during pregnancy, for example by analyzing, for example sequencing, embryo-derived cells in the mothers blood. Other relevant gene signatures—blood type 0, rhesus negativity—will also be determined. Then a haplotype- and/or bloodtype-matched human subject in need of cell therapy will be identified in a database.

Upon birth at term by either cesarian section or regular birth by labour the placenta is then used for isolation of the desired embryo-derived somatic cell type, such as embryo-derived somatic mononuclear phagocytic cells, and in particular the embryo-derived human placental macrophages. After cell isolation from the placenta, isolated cells are then prepared and/or further modified for cell therapy of a preferably immunologically matching recipient.

The present inventors have found that surprisingly embryo-derived somatic cells from human term placenta, such as embryo-derived somatic mononuclear phagocytic cells isolated from human term placenta, such as embryo-derived human placental macrophages isolated from human term placenta, are effective at treating a condition in a subject in need of cell therapy, such as the physiological defects observable in a preclinical mouse model of pulmonary alveolar proteinosis.

By isolating macrophages from term placenta, the present invention provides macrophages which are immunologically very young and have a high potential for regenerative therapies, as they are functionally close to embryonic macrophages. But while the access to embryonic macrophages for cell therapy is unethical as it would involve the death of the embryo, macrophages from term placenta don't have this ethical problem as they are derived from a tissue that is typically discarded after birth or after cesarian section. Thus, the present invention has the advantage of providing human macrophages which are as close as possible to embryonic macrophages, but which macrophages are provided in a way that does not raise the same ethical concerns. Thus, the present invention also relates to human placental macrophages for use in regenerative medicine.

The invention also relates to the use of placental macrophages for autologous cell therapy. The placental macrophages can of course also be administered to the corresponding newborn, for example in those cases where the newborn is suffering from a defect in macrophage function. In particular premature infants, such as newborn children who were born before the 37th week of pregnancy and/or infants who are very small and have a weight at birth of at most 2500 g, often suffer from functional deficiencies such as infant respiratory distress syndrome. In infant respiratory distress syndrome there is a deficiency of pulmonary surfactant production, often leading to the collapse of alveoli. Premature infants have very low numbers of alveolar macrophages, which are involved in the regulation of surfactant production, and intratracheal administration of placental macrophages is predicted to restore the regulation of surfactant production. Therefore, the present invention also relates to human placental macrophages for use in the treatment of infant respiratory distress syndrome, in particular wherein the use is autologous.

As a further surprise, the present inventors have identified two separate populations of human embryo-derived placental macrophages, which are both useful in cell therapy. The invention therefore also relates to the two human embryo-derived placental macrophage populations and to their use in medicine.

The present invention relates to a first human placental macrophage, wherein the macrophage is characterized by (Expression pattern 1) very high mRNA expression of RNASE1, LGMN and C1QB. Alternatively, the first human placental macrophage can be characterized by at least high mRNA expression of MRC1, PLTP and MAF (Expression pattern 2). Alternatively, the first human placental macrophage can be characterized by at least medium mRNA expression of SIGLEC11 (Expression pattern 3). Alternatively, the first human placental macrophage can be characterized by at most high mRNA expression of CFP, APOBEC3A and CD55 (Expression pattern 4). Alternatively, the first human placental macrophage can be characterized by at most medium mRNA expression of $100A1, SERPINB2, LRG1 and PTX3 (Expression pattern 5). Alternatively, the first human placental macrophage can be characterized by at most low mRNA expression of PTGES (Expression pattern 6). Alternatively, the first human placental macrophage can be characterized by no mRNA expression of IL18RAP and PPBP (Expression pattern 7).

More specifically the first human placental macrophage of the invention can be characterized by combinations of two, three, four, five, six or all seven of the above described expression patterns (EP) 1 to 7, such as EP1+EP2, EP1+EP2+EP3, EP1+EP2+EP3+EP4, EP1+EP2+EP3+EP4+EP5, EP1+EP2+EP3+EP4+EP5+EP6 or EP1+EP2+EP3+EP4+EP5+EP6+EP7, to name just one example each for the combinations of two, three, four, five, six or all seven of the above described expression patterns (EP). The skilled person will understand that this passage discloses all possible permutations for the combinations of two, three, four, five, six or all seven of the above-described expression patterns (EP), such as—now using random examples—also the combination of EP3 with EP6 as an example for a combination of two expression patterns, or such as the combination of EP2 with EP5 and EP7, as a random example for a combination of three expression patterns. Further specific examples of combinations of expression patterns are EP1+EP3, EP1+EP4, EP1+EP5, EP1+EP6, EP1+EP7, EP2+EP3, EP2+EP4, EP2+EP5, EP2+EP6, EP2+EP7, EP3+EP4, EP3+EP5, EP3+EP6, EP3+EP7, EP4+EP5, EP4+EP6, EP4+EP7, EP5+EP6, EP5+EP7, EP6+EP7, EP1+EP2+EP4, EP1+EP2+EP5, EP1+EP2+EP6, EP1+EP2+EP7, EP1+EP3+EP4, EP1+EP3+EP5, EP1+EP3+EP6, EP1+EP3+EP7, EP1+EP4+EP5, EP1+EP4+EP6, EP1+EP4+EP6, EP1+EP5+EP6, EP1+EP5+EP7 and EP1+EP6+EP7.

Alternatively, the first human placental macrophage can be characterized by surface markers. The present invention also relates to the first human placental macrophage, wherein the macrophage is characterized by the presence of the surface markers CD45, CD14, LYVE1 and SIGLEC1 (Surface marker pattern SM1). Alternatively, the first human placental macrophage can be characterized by

by the absence of the surface markers CD3, CD19, CD56, CD66b and CCR2 (SM2). Alternatively, the first human placental macrophage can be characterized by the presence of the surface marker MRC1 (SM3). Alternatively, the first human placental macrophage can be characterized by the presence of surface marker FOLR2 (SM4).

More specifically the first human placental macrophage of the invention can be characterized by combinations of two, three or all four of the above-described surface marker patterns (SM) 1 to 4, such as SM1+SM2, SM1+SM2+SM3 or SM1+SM2+SM3+SM4, to name just one example each for the combinations of two, three or all four of the above-described surface marker patterns (SM). The skilled person will understand that this passage discloses all possible permutations for the combinations of two, three or all four of the above-described expression patterns (SM), such as—now using random examples—also the combination of SM3 with SM4 as an example for a combination of two expression patterns, or such as the combination of SM2 with SM3 and SM4, as a random example for a combination of three expression patterns. Further specific examples of combinations of expression patterns arc SM1+SM3, SM1+SM4, SM2+SM3, SM2+SM4, SM3+SM4, SM1+SM2+SM4 and SM1+SM3+SM4.

The skilled person will understand that the first human placental macrophage can also be characterized by the combination of expression patterns and surface markers, such as, for example EP2+SM2 or EP6+SM4, to chose just random examples for the combination of one expression pattern with one surface marker pattern. The skilled person will understand that the first human placental macrophage can also be characterized by the combination of the above-described combinations of expression patterns with combinations of the above-described surface marker patterns, such as, for example EP1+EP3+EP4 combined with SM2+SM4 or EP1+EP2+EP7 combined with SM1+SM2, to choose just random examples for the combination of one combination of expression patterns with one combination of surface marker patterns.

Depending on the intended specific medical use, the human macrophage of the invention and/or the human macrophages comprised by the collection of human macrophages of the invention may be further genetically modified.

The present invention also relates to a collection of cells comprising the first human placental macrophages, wherein the collection comprises at least 106 cells, and preferably 106 first macrophages. Preferably the collection of cells comprises at least 108 cells, and preferably 108 macrophages, such as from 108 to 1012 cells, and preferably 108 to 1012 first macrophages. The collection of cells may also comprise from 109 to 1011 cells, and preferably from 109 to 1011 first macrophages.

The collection of cells comprising the first human placental macrophages can also comprise cells other than first human placental macrophages, but preferably at least 60% of the cells are first human placental macrophages, more preferably at least 80% and even more preferably at least 90% of the cells are first human placental macrophages, such as in a collection of cells consisting essentially of first human placental macrophages.

The present invention relates to a second human placental macrophage, wherein the macrophage is characterized by Human placental macrophage, wherein the macrophage is characterized by very high mRNA expression of SERPINB2, CFP, APOBEC3A and CD55 (EP1a). Alternatively, the second human placental macrophage can be characterized by at least high mRNA expression of S100A12, PTGES, LRG1 and PTX3 (EP2a). Alternatively, the second human placental macrophage can be characterized by at least low mRNA expression of IL18RAP and PPBP (EP3a). Alternatively, the second human placental macrophage can be characterized by at most high mRNA expression of PLTP, RNASE1, LGMN and C1QB (EP4a). Alternatively, the second human placental macrophage can be characterized by at most medium mRNA expression of MRC1 and MAF (EP5a). Alternatively, the second human placental macrophage can be characterized by at most low mRNA expression of CCL18, ADORA3A and SIGLEC11 (EP6a).

More specifically the second human placental macrophage of the invention can be characterized by combinations of two, three, four, five or all six of the above-described expression patterns (EP) 1a to 6a, such as EP1a+EP2a, EP1a+EP2a+EP3a, EP1a+EP2a+EP3a+EP4a, EP1a+EP2a+EP3a+EP4a+EP5a or EP1a+EP2a+EP3a+EP4a+EP5a+EP6a to name just one example each for the combinations of two, three, four, five or all six of the above-described expression patterns (EP). The skilled person will understand that this passage discloses all possible permutations for the combinations of two, three, four, five or all six of the above-described expression patterns (EP), such as—now using random examples—also the combination of EP3a with EP6a as an example for a combination of two expression patterns, or such as the combination of EP2a with EP4a and EP6a, as a random example for a combination of three expression patterns. Further specific examples of combinations of expression patterns are EP1a+EP3a, EP1a+EP4a, EP1a+EP5a, EP1a+EP6a, EP2a+EP3a, EP2a+EP4a, EP2a+EP5a, EP2a+EP6a, EP3a+EP4a, EP3a+EP5a, EP3a+EP6a, EP4a+EP5a, EP4a+EP6a, EP5a+EP6a, EP1a+EP2a+EP4a, EP1a+EP2a+EP5a, EP1a+EP2a+EP6a, EP1a+EP3a+EP4a, EP1a+EP3a+EP5a, EP1a+EP3a+EP6a, EP1a+EP4a+EP5a, EP1a+EP4a+EP6a, EP1+EP4a+EP6a and EP1a+EP5a+EP6a.

Alternatively, the second human placental macrophage can be characterized by surface markers. The present invention also relates to the second human placental macrophage, wherein the macrophage is characterized by the presence of the surface markers CD45, CD14 and CD89, and the absence of the surface markers LYVE1 and SIGLEC1 (surface marker pattern SM1a). Alternatively, the second human placental macrophage can be characterized by the absence of the surface markers CD3, CD19, CD56, CD66b and CCR2 (SM2a). Alternatively, the second human placental macrophage can be characterized by the absence of the surface marker MRC1 (SM3a).

More specifically the second human placental macrophage of the invention can be characterized by combinations of two or all three of the above-described surface marker patterns (SM) 1a to 3a, such as SM1a+SM2a, SM1a+SM3a, SM2a+SM3a and SM1a+SM2a+SM3a.

The skilled person will understand that the first human placental macrophage can also be characterized by the combination of expression patterns and surface markers, such as, for example EP2a+SM2a or EP6a+SM3a, to choose just random examples for the combination of one expression pattern with one surface marker pattern. The skilled person will understand that the first human placental macrophage can also be characterized by the combination of the above-described combinations of expression patterns with combinations of the above-described surface marker patterns, such as, for example EP1a+EP3a+EP4a combined with SM2a+SM3a or EP1a+EP2a+EP5a combined with SM1a+SM3a, to choose just random examples for the combination of one combination of expression patterns with one combination of surface marker patterns.

Depending on the intended specific medical use, the human macrophage of the invention and/or the human macrophages comprised by the collection of human macrophages of the invention may be further genetically modified.

The present invention also relates to a collection of cells comprising the second human placental macrophages, wherein the collection comprises at least 106 cells, and preferably 106 second macrophages. Preferably the collection of cells comprises at least 108 cells, and preferably 108 second macrophages, such as from 108 to 1012 cells, and preferably 108 to 1012 second macrophages. The collection of cells may also comprise from 109 to 1011 cells, and preferably from 109 to 1011 second macrophages.

The collection of cells comprising the second human placental macrophages can also comprise cells other than second human placental macrophages, but preferably at least 60% of the cells are second human placental macrophages, more preferably at least 80% and even more preferably at least 90% of the cells are second human placental macrophages, such as in a collection of cells consisting essentially of second human placental macrophages.

The present invention also relates to human macrophages obtainable by deleting the transcription factors MAF and MAFB in the first or the second human placental macrophage of the invention. It is preferred that the deletion of MAFB is effected by deleting at least 50 base pairs within the region on chromosome 22 from 40685700 to 40689300 on both alleles of chromosome 22. In particular the deletion of MAFB can be effected by deleting from 500 base pairs to 3000 base pairs within said region.

It is also preferred that the deletion of MAF is effected by deleting at least 50 base pairs within the region on chromosome 16 from 79593600 to 79600900 on both alleles of chromosome 16. In particular the deletion of MAF can be effected by deleting from 500 base pairs to 3000 base pairs. Preferably other than MAF and MAFB no further transcription factors are deleted. Such macrophages have the additional advantage that they have an increased proliferative potential and can be expanded ex vivo, thereby allowing the generation of higher cell numbers ex vivo, but without leading to a transformation of the manipulated cells into tumorigenic cells.

The deletion of MAF and MAFB in the above-mentioned regions can, for example, be effected by using the CRISPR/Cas9 system. Cas9 and guide RNAs can be expressed by publicly and commercially available DNA expression plasmids well known in the art (for example Santa Cruz Sc-418922, https://www.scbt.com/de/p/control-crispr-cas9-plasmid). DNA expression plasmids can be introduced into the first or the second human placental macrophages of the invention by electroporation or lipid-based transfection protocols well known to the art. Cas9 and gRNA can also be introduced into the first or the second human placental macrophages of the invention as a ribonucleic/protein complex by electroporation. Cas9/gRNA mediated gene editing has been demonstrated in mononuclear phagocytes using such methods (Zhag et al. (2020); Wang et al. (2018)) and in human CD34+ hematopoietic stem and progenitor cells differentiating to macrophages (Scharenberg et al. (2020)). Freund ct al. (2020) described a preferred method for effecting gene knockouts in myeloid cells by non-viral delivery of CRISPR-Cas9 which uses nucleofection-based delivery of Cas9-ribonucleoprotein particles. MafB and cMaf genes could also be deleted utilizing engineered site directed recombinases (Lansing et al. (2020); Karpinsky et al. (2016)), which can be introduced by methods known to the art, such as electroporation of the protein, coding mRNA or DNA expression plasmids, and which have been used for gene editing in mononuclear phagocytes (Shi et al. (2018)).

The present invention also relates to a collection of cells comprising the human macrophages obtainable by deleting the transcription factors MAF and MAFB in the first or the second human placental macrophage of the invention, wherein the collection comprises at least 106 cells, and preferably 106 human macrophages obtainable by deleting the transcription factors MAF and MAFB in the first or the second human placental macrophage of the invention. Preferably the collection of cells comprises at least 108 cells, and preferably 108 second macrophages, such as from 108 to 1012 cells, and preferably 108 to 1012 human macrophages obtainable by deleting the transcription factors MAF and MAFB in the first or the second human placental macrophage of the invention. The collection of cells may also comprise from 109 to 1011 cells, and preferably from 109 to 1011 human macrophages obtainable by deleting the transcription factors MAF and MAFB in the first or the second human placental macrophage of the invention.

The collection of cells comprising the human macrophages obtainable by deleting the transcription factors MAF and MAFB in the first or the second human placental macrophage of the invention can also comprise cells other than human macrophages obtainable by deleting the transcription factors MAF and MAFB in the first or the second human placental macrophage of the invention, but preferably at least 60% of the cells are human macrophages obtainable by deleting the transcription factors MAF and MAFB in the first or the second human placental macrophage of the invention, more preferably at least 80% and even more preferably at least 90% of the cells are human macrophages obtainable by deleting the transcription factors MAF and MAFB in the first or the second human placental macrophage of the invention, such as in a collection of cells consisting essentially of human macrophages obtainable by deleting the transcription factors MAF and MAFB in the first or the second human placental macrophage of the invention.

The present invention also relates to the use of the first and/or the second macrophage of the invention for use as a medicament. The present invention also relates to the use of the human macrophages obtainable by deleting the transcription factors MAF and MAFB in the first or the second human placental macrophage of the invention for use as a medicament. The present invention also relates to the use of a collection of first human placental macrophages for use as a medicament. The present invention also relates to the use of a collection of second human placental macrophages for use as a medicament. The present invention also relates to the use of a collection of the human macrophages obtainable by deleting the transcription factors MAF and MAFB in the first or the second human placental macrophage of the invention for use as a medicament.

The invention also provides a pharmaceutical composition comprising a collection of the first and/or second human placental macrophages of the invention. The invention also provides a pharmaceutical composition comprising a collection of the human macrophages obtainable by deleting the transcription factors MAF and MAFB in the first or the second human placental macrophage of the invention. Pharmaceutically acceptable delivery methods and formulations for cell therapy have been described in the art. Cells can be suspended in a pharmaceutically acceptable carrier, such as a buffer, e.g. PBS or PBS/EDTA supplemented with about 20% human serum albumin, or citrate plasma, or Plasmalyte-A pH 7.4 (Baxter; supplemented with about 2% HSA). The pharmaceutically acceptable carrier for the macrophages of the invention is compatible with survival of the cells. It may comprise physiological concentrations of NaCl.

The first and/or the second human placental macrophages or the collection of first and/or second human placental macrophages of the invention can be used in therapies where macrophage cell therapy has provided successful results. Also the human macrophages obtainable by deleting the transcription factors MAF and MAFB in the first or the second human placental macrophage of the invention can be used in therapies where macrophage cell therapy has provided successful results. Non-limiting examples are the treatment of a disease selected from the group consisting of a cancer, an immune-deficiency, a chronic or an acute injury, such as central nervous system injury—for example spinal cord injury-acute injury such as ischemic stroke, hepatic injury or myocardial infarction, a wound, such as a chronic wound, a degenerative disease, an autoimmune disease, such as type 1 diabetes and Crohn's disease, rheumatoid arthritis or osteoarthritis, a chronic inflammatory disease, atherosclerosis, poly- and ostco-arthritis, osteoporosis, an infectious disease (e.g. infections by virus, or bacteria), and a metabolic disease.

The present invention also relates to a method for the isolation of human placental macrophages from human term placenta, comprising the steps of
a. obtaining villous-containing tissue from the placenta;
b. disintegrating said villous-containing tissue;
c. separating human placental macrophages from debris, cytotrophoblasts and fibroblasts to obtain a cell population comprising placental macrophages; and
d. isolating human placental macrophages by selecting cells characterized by the presence of the surface markers CD45, CD14, LYVE1 and SIGLEC1 or by selecting cells characterized by the presence of the surface markers CD45, CD14 and CD89, and the absence of the surface markers LYVE1 and SIGLEC1.

The present invention also relates to a method for the isolation of human placental macrophages from human term placenta, comprising the steps of
a. obtaining villous-containing tissue from the placenta;
b. disintegrating said villous-containing tissue;
c. separating human placental macrophages from debris, cytotrophoblasts and fibroblasts to obtain a cell population comprising placental macrophages; and
d. isolating human placental macrophages by selecting cells characterized by the presence of the surface markers CD45, CD14, MRC1 and FOLR2 (first human placental macrophage population) or by selecting cells characterized by the presence of the surface markers CD45, CD14 and CD89, and the absence of the surface markers MRC1 and SIGLEC1 (second human placental macrophage population) or the absence of the surface marker MRC1 and lower expression of the surface marker FOLR2 when compared to the first macrophage population.

Placentas are mechanically taken apart and the villous tissue, which comprises the first and second embryo-derived placental macrophages, is separated mechanically from membrane tissue. Then the villous tissue is mechanically disintegrated and/or connecting tissue within the villous enzymatically digested in order to disintegrate the villous preferably into single cells. Then a particle size separation method, such as sieving, can be used to remove remaining fibrous material or clumps of cells or tissue, so that cell types can then be separated based on their densities and/or by methods which purify based on immunological properties of the cells, e.g. based on differences of their cell surface markers. Such an immunopurification can comprise one or more negative selection step(s) where cells having surface markers which are specific for cytotrophoblasts and/or fibroblasts are removed. The remaining collection of cells, which comprises the first and the second placental macrophages of the invention, can then be further separated with the help of suitable antibodies against cell-type specific surface markers by methods such as automated cell sorting. Automatic cell sorting then also allows the preparation and separation of the first and second human placental macrophages of the invention, for example by selecting cells in step d) which are also characterized by the absence of the surface markers CD3, CD19, CD56, CD66b and CCR2. An automatic cell sorting step where gating is for cells where the surface markers CD3, CD19, CD56, CD66b and CCR2 are absent helps to eliminate possibly contaminating T-cells, B-cells, NK-cells, granulocytes and monocytes.

The invention further relates to the following embodiments
1. Human placental macrophage, wherein the macrophage is characterized by very high mRNA expression of RNASE1, LGMN and C1QB.
2. The human placental macrophage according to item 1, characterized by at least high mRNA expression of MRC1, PLTP and MAF.
3. The human placental macrophage according to any one of items 1 to 2, characterized by at least medium mRNA expression of SIGLEC11.
4. The human placental macrophage according to any one of items 1 to 3, characterized by at most high mRNA expression of CFP, APOBEC3A and CD55.
5. The human placental macrophage according to any one of items 1 to 4, characterized by at most medium mRNA expression of S100A1, SERPINB2, LRG1 and PTX3.
6. The human placental macrophage according to any one of items 1 to 5, characterized by at most low mRNA expression of PTGES.
7. The human placental macrophage according to any one of items 1 to 6, characterized by no mRNA expression of IL18RAP and PPBP.
8. The human placental macrophage according to any one of items 1 to 7, wherein the macrophage is characterized by the presence of the surface markers CD45, CD14, LYVE1 and SIGLEC1.
9. The human placental macrophage according to any one of items 1 to 8, wherein the macrophage is characterized by the absence of the surface markers CD3, CD19, CD56, CD66b and CCR2.
10. The human placental macrophage according to any one of items 1 to 9, wherein the macrophage is characterized by the presence of the surface marker MRC1.
11. The human placental macrophage according to any one of items 1 to 10, wherein the macrophage is characterized by the presence of surface marker FOLR2.
12. Human placental macrophage, wherein the macrophage is characterized by very high mRNA expression of SERPINB2, CFP, APOBEC3A and CD55.
13. The human placental macrophage according to item 12, characterized by at least high mRNA expression of S100A12, PTGES, LRG1 and PTX3.
14. The human placental macrophage according to any one of items 12 to 13, characterized by at least low mRNA expression of IL18RAP and PPBP.
15. The human placental macrophage according to any one of items 12 to 14, characterized by at most high mRNA expression of PLTP, RNASE1, LGMN and CQB.
16. The human placental macrophage according to any one of items 12 to 15, characterized by at most medium mRNA expression of MRC1 and MAF.
17. The human placental macrophage according to any one of items 12 to 16, characterized by at most low mRNA expression of CCL18, ADORA3A and SIGLEC11.
18. The human placental macrophage according to any one of items 12 to 17, characterized by the presence of the surface markers CD45, CD14 and CD89, and the absence of the surface markers LYVE1 and SIGLEC1.
19. The human placental macrophage according to any one of items 12 to 18, wherein the macrophage is characterized by the absence of the surface markers CD3, CD19, CD56, CD66b and CCR2.
20. The human placental macrophage according to any one of items 12 to 19, wherein the macrophage is characterized by the absence of the surface marker MRC1.
21. Human placental macrophage obtainable by deleting the genes MAF and MAFB in a human placental macrophage according to any one of items 1 to 11 or 12 to 20.
22. The human placental macrophage according to item 21, wherein the deletion of MAFB is effected by deleting at least 50 base pairs within the region on chromosome 22 from 40685700 to 40689300 on both alleles of chromosome 22.
23. The human placental macrophage according to item 22, wherein the deletion of MAFB is effected by deleting from 500 base pairs to 3000 base pairs.
24. The human placental macrophage according to item 21, wherein the deletion of MAF is effected by deleting at least 50 base pairs within the region on chromosome 16 from 79593600 to 79600900 on both alleles of chromosome 16.
25. The human placental macrophage according to item 24, wherein the deletion of MAF is effected by deleting from 500 base pairs to 3000 base pairs.
26. The human placental macrophage according to any one of items 21 to 25, wherein MAF and MAFB are the only transcription factors which are deleted.
27. The human placental macrophage according to any one of items 1 to 26, wherein the macrophage does not comprise the blood-type antigens A and B.
28. The human placental macrophage according to any one of items 1 to 27, wherein the macrophage does not comprise the Rhesus D-antigen.
29. The human placental macrophage according to any one of items 1 to 28, wherein the macrophage is homozygous for the genes encoding the human leukocyte antigens HLA-A, HLA-B or HLA-DR.
30. The human placental macrophage according to any one of items 1 to 29, wherein the macrophage is homozygous for the genes encoding the human leukocyte antigens HLA-A, HLA-B and HLA-DR.
31. The human placental macrophage according to any one of items 1 to 30, wherein the macrophage is homozygous for the allele HLA-A1.
32. The human placental macrophage according to any one of items 1 to 30, wherein the macrophage is homozygous for the allele HLA-A2.
33. The human placental macrophage according to any one of items 1 to 30, wherein the macrophage is homozygous for the allele HLA-A3.
34. The human placental macrophage according to any one of items 1 to 30, wherein the macrophage is homozygous for the allele HLA-A29 (19).
35. The human placental macrophage according to any one of items 1 to 30, wherein the macrophage is homozygous for the allele HLA-All.
36. The human placental macrophage according to any one of items 1 to 30, wherein the macrophage is homozygous for the allele HLA-A24.
37. The human placental macrophage according to any one of items 1 to 30, wherein the macrophage is homozygous for the allele HLA-B8.
38. The human placental macrophage according to any one of items 1 to 30, wherein the macrophage is homozygous for the allele HLA-B44 (12).
39. The human placental macrophage according to any one of items 1 to 30, wherein the macrophage is homozygous for the allele HLA-B7.
40. The human placental macrophage according to any one of items 1 to 30, wherein the macrophage is homozygous for the allele HLA-B62 (15).
41. The human placental macrophage according to any one of items 1 to 30, wherein the macrophage is homozygous for the allele HLA-B57 (17).
42. The human placental macrophage according to any one of items 1 to 30, wherein the macrophage is homozygous for the allele HLA-B35.
43. The human placental macrophage according to any one of items 1 to 30, wherein the macrophage is homozygous for the allele HLA-B40.
44. The human placental macrophage according to any one of items 1 to 30, wherein the macrophage is homozygous for the allele HLA-B46.
45. The human placental macrophage according to any one of items 1 to 30, wherein the macrophage is homozygous for the allele HLA-DR17 (3).
46. The human placental macrophage according to any one of items 1 to 30, wherein the macrophage is homozygous for the allele HLA-DR4.
47. The human placental macrophage according to any one of items 1 to 30, wherein the macrophage is homozygous for the allele HLA-DR15 (2).
48. The human placental macrophage according to any one of items 1 to 30, wherein the macrophage is homozygous for the allele HLA-DR7.
49. The human placental macrophage according to any one of items 1 to 30, wherein the macrophage is homozygous for the haplotype HLA-A1, HLA-B8, HLA-DR17 (3).
50. The human placental macrophage according to any one of items 1 to 30, wherein the macrophage is homozygous for the haplotype HLA-A2, HLA-B44 (12), HLA-DR4.
51. The human placental macrophage according to any one of items 1 to 30, wherein the macrophage is homozygous for the haplotype HLA-A3, HLA-B7, HLA-DR15 (2).
52. The human placental macrophage according to any one of items 1 to 30, wherein the macrophage is homozygous for the haplotype HLA-A2, HLA-B7, HLA-DR15 (2).
53. The human placental macrophage according to any one of items 1 to 30, wherein the macrophage is homozygous for the haplotype HLA-A2, HLA-B44 (12), HLA-DR7.
54. The human placental macrophage according to any one of items 1 to 30, wherein the macrophage is homozygous for the haplotype HLA-A2, HLA-B62 (15), HLA-DR4.
55. The human placental macrophage according to any one of items 1 to 30, wherein the macrophage is homozygous for the haplotype HLA-A1, HLA-B57 (17), HLA-DR7.
56. The human placental macrophage according to any one of items 1 to 30, wherein the macrophage is homozygous for the haplotype HLA-A3, HLA-B35, HLA-DR1.
57. The human placental macrophage according to any one of items 1 to 30, wherein the macrophage is homozygous for the haplotype HLA-A29 (19), HLA-B44 (12), HLA-DR7.
58. A collection of cells comprising human placental macrophages according to any one of items 1 to 57, wherein the collection comprises at least 106 macrophages.
59. The collection of cells comprising human placental macrophages according to item 58, wherein the collection comprises at least 108 macrophages.
60. The collection of cells comprising human placental macrophages according to item 59, wherein the collection comprises from 108 to 1012 macrophages.
61. The collection of cells comprising human placental macrophages according to item 60, wherein the collection comprises from 109 to 1011 macrophages.
62. The collection of cells comprising human placental macrophages according to any one of items 58 to 61, wherein at least 60% of the cells are human placental macrophages according to any one of items 1 to 11.
63. The collection of cells comprising human placental macrophages according to any one of items 58 to 61, wherein at least 80% of the cells are human placental macrophages according to any one of items 1 to 11.
64. The collection of cells comprising human placental macrophages according to any one of items 58 to 61, wherein at least 90% of the cells are human placental macrophages according to any one of items 1 to 11.
65. The collection of cells comprising human placental macrophages according to any one of items 58 to 61, wherein at least 60% of the cells are human placental macrophages according to any one of items 12 to 20.
66. The collection of cells comprising human placental macrophages according to any one of items 58 to 61, wherein at least 80% of the cells are human placental macrophages according to any one of items 12 to 20.
67. The collection of cells comprising human placental macrophages according to any one of items 58 to 61, wherein at least 90% of the cells are human placental macrophages according to any one of items 12 to 20.
68. The collection of cells comprising human placental macrophages according to any one of items 58 to 61, wherein the human placental macrophages are human placental macrophages according to any one of items 8 to 13.
69. The collection of cells comprising human placental macrophages according to any one of items 58 to 61, wherein the human placental macrophages are human placental macrophages according to any one of items 21 to 57.
70. Human placental macrophages according to any one of items 1 to 57 or a collection of human placental macrophages according to any one of items 58 to 69 for use as a medicament.
71. The human placental macrophages or the collection of human macrophages for use as a medicament according to item 70, wherein the human placental macrophages are derived from a term placenta.
72. Human placental macrophages according to any one of items 1 to 57 or a collection of human placental macrophages according to any one of items 58 to 69 for use as a medicament for the treatment of a disease selected from the group consisting of a cancer, an immune-deficiency, a chronic or an acute injury, such as central nervous system injury—for example spinal cord injury-acute injury such as ischemic stroke, hepatic injury or myocardial infarction, a wound, such as a chronic wound, a degenerative disease, an autoimmune disease, such as type 1 diabetes and Crohn's disease, rheumatoid arthritis or osteoarthritis, a chronic inflammatory disease, atherosclerosis, poly- and osteo-arthritis, osteoporosis, an infectious disease (e.g. infections by virus, or bacteria), and a metabolic disease.
73. A method for the isolation of human placental macrophages from human term placenta, comprising the steps of
   a. obtaining villous-containing tissue from the placenta;
   b. disintegrating said villous-containing tissue;
   c. separating human placental macrophages from debris, cytotrophoblasts and fibroblasts to obtain a cell population comprising placental macrophages; and
   d. isolating human placental macrophages by selecting cells characterized by the presence of the surface markers CD45, CD14, LYVE1 and SIGLEC1 or by selecting cells characterized by the presence of the surface markers CD45, CD14 and CD89, and the absence of the surface markers LYVE1 and SIGLEC1.
74. The method of item 73, wherein in step d) cells are selected which are characterized by the absence of the surface markers CD3, CD19, CD56, CD66b and CCR2.
75. The method of items 73 to 74, wherein step a) comprises mechanical separation of villous tissue from membrane tissue.
76. The method of any one of items 73 to 75, wherein step b) comprises mechanical disintegration of villous tissue.
77. The method of any one of items 73 to 76, wherein step b) comprises enzymatic digestion of connecting tissue.
78. The method of any one of items 73 to 77, wherein step c) comprises sieving.
79. The method of any one of items 73 to 78, wherein step c) comprises separating cell types based on their densities.
80. The method of any one of items 73 to 79, wherein step c) comprises immunopurification.
81. The method of item 80, wherein immunopurification comprises a negative selection of cell having surface markers which are specific for cytotrophoblasts and/or fibroblasts.
82. The method of any one of items 73 to 81, wherein step d) comprises automated cell sorting.
83. Use of human term placenta in the preparation of human mononuclear phagocytic cells, wherein the human mononuclear phagocytic cells are for use as a cell therapy.
84. The use of item 83, wherein the human mononuclear phagocytic cells are macrophages.
85. The use of item 84, wherein the macrophages are human placental macrophages according to any one of items 1 to 57.
86. Use of human term placenta as a source of haplotype-matched placental cells for use in cell therapy.
87. The use of item 86, wherein the placental cells are characterized by any one of the immunological features or combination of immunological features described for macrophages in any one of items 27 to 57.
88. The use of item 87, wherein the placental cells are macrophages.
89. The use according to any one of items 86 to 88, wherein the placental cells are genotype-matched.
90. The use according to any one of items 86 to 89, wherein the fetal genotype with regard to HLA-A, HLA-B and HLA-DR is determined prior to birth.
91. The use according to any one of items 86 to 90, wherein the fetal blood type is determined prior to birth.
92. The use according to any one of items 86 to 91, wherein a haplotype-matched human subject in need of cell therapy is identified in a database.
93. The use according to any one of items 86 to 92, wherein a genotype-matched human subject in need of cell therapy is identified in a database.
94. The use according to any one of items 91 to 93, wherein a bloodtype-matched human subject in need of cell therapy is identified in a database.
95. The use according to any one of items 91 to 94, wherein a haplotype- and bloodtype-matched human subject in need of cell therapy is identified in a database.
96. The use according to any one of items 91 to 95, wherein a genotype- and bloodtype-matched human subject in need of cell therapy is identified in a database.
97. Method of treating a patient in need of cell therapy, wherein the method comprises the steps
   a. identifying the haplotype of a fetus with regard to HLA-A, HLA-B and HLA-DR prior to birth;
   b. identifying a haplotype-matched human subject in need of cell therapy in a database;
   c. isolating haplotype-matched cells from the placenta of said fetus identified in step a); and
   d. preparing a pharmaceutical composition comprising said isolated cells.
98. The method of item 97, wherein the fetus and the human subject to be treated are matched with regard to blood-type.
99. The method of any one of items 97 to 98, wherein the fetus and the human subject to be treated are genotype-matched with regard to HLA-A, HLA-B and HLA-DR.
100. The method according to any one of items 97 to 99, wherein the cells are human placental macrophages according to any one of items 1 to 57.
101. The method according to any one of items 97 to 100, wherein the subject in need of cell therapy is suffering from a disorder in macrophage function.
102. The method according to item 101, wherein the disorder in macrophage function is an increased susceptibility to infections with intracellular organisms, in particular to infection with mycobacteria.
103. The use according to any one of items 86 to 89, wherein the use is autologous cell therapy.
104. The use according to item 103, wherein placental macrophages are for use in the treatment of a macrophage deficiency of the corresponding newborn.
105. The use according to item 104, wherein the newborn was born before the 37th week of pregnancy.
106. The use according to any one of items 104 to 105, wherein the newborn had a weight at birth of at most 2500 g.
107. The use according to any one of items 104 to 106, wherein the newborn is suffering from infant respiratory distress syndrome.
108.Human placental macrophages, in particular the human placental macrophages according to any one of items 1 to 57 or the collection of human placental macrophages according to any one of items 58 to 69, for use in the treatment of infant respiratory distress syndrome.
109. The use according to item 108, wherein the use is autologous.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Handbook of Experimental Immunology” (Weir, 1997); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques may be considered in making and practicing the invention.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Materials and Methods

### Human Samples

All tissue samples used were obtained with written consent from study participants. Peripheral blood and placental tissues were obtained from healthy women (carrying a male fetus, as this helps to distinguish and differentiate the fetal-derived cells from the mother and thus makes it easier to isolate or identify fetal derived cells. Example: If the mother cells are HLA-A3+ and the fetal-derived cells are HLA-A2+ then they can be easily distinguished based on surface markers) with normal pregnancies undergoing elective C-sections at term/third-trimester (38-40 wk; n=34). Maternal cells and fetal cells of a male fetus can be distinguished in sequencing experiments based on the presence or absence of X and Y-chromosome specific transcripts. Infection was excluded on the basis of standard clinical criteria (absence of fever, uterine tenderness, maternal/fetal tachycardia, foul vaginal discharge). Approval for this study was obtained from the Technical University Dresden Research Ethics Committee.

### Tissue Processing

We wanted to first gain an overview of all immune cells which are located in the placenta, and—in a broader sense—which are involved in the communication between the mother's and the baby's immune systems. To that end cells were isolated from the placenta, from the decidua, from maternal blood and from cord blood. This would allow us to compare various cell types, which can be found in the placenta, with one another.

Placentas from uncomplicated term pregnancies were brought to the laboratory within 30 min following elective cesarean section without labor at UKD (Uniklinikum Dresden). The decidua basalis was removed first using scissors. The placental villi were collected as pieces using scissors, washed several times with PBS, minced and digested with 0.25% Trypsin/0.02% EDTA/0.2% DNase I at 37° C. with stirring for 10 min. The resulting smaller tissue clumps were passed through a sterile muslin gauze, and washed with DMEM-F12+NCS+DNase I to stop the digestion process. The filtrate containing single cells was stored on ice. The smaller tissue clumps which were left on the gauze were digested in 1 mg/ml collagenase V (Sigma-Aldrich), supplemented with 0.1 mg/ml DNase I (Sigma) for 75 min at 37° C. in a shaking waterbath in order to improve the overall yield. The cell suspension resulting from that collagenase digest was passed through a sterile muslin gauze, filtered through 100 μm first, then through 70 μm, and the cell-containing filtrate was washed with PBS by a suspension/centrifugation step: cell suspensions from the filtrates of both the trypsin and collagenase digests were pooled and pelleted by centrifuging for 5 min at 1500 rpm (rotations per minute; equivalent to 305 x g), resuspended in PBS and combined. Cells were layered onto a Percoll gradient (GE Healthcare) (70%-50%-25%) and spun for 30 min without brake at 2,000 rpm. The leukocyte layers at the interfaces between 25% & 50% and between 50% & 70% were collected and washed in PBS (suspension in PBS and repelleting). This protocol is based on Tang et al., 2011 (PMID: 21545365), but includes the described adaptations and modifications.

For comparative reasons and in order to understand the immune cells which are present at the interface between embryo and mother in their entirety, leukocytes were also isolated from the decidua and blood. Decidual samples and blood samples were processed separately, but simultaneously.

Decidual tissues are formed from maternal cells and were thus used as control cells in single cell RNAseq experiments. The decidua contains maternal macrophages. The decidual tissue was digested in 1 mg/ml collagenase V (Sigma-Aldrich), supplemented with 0.1 mg/ml DNase I (Sigma) for 75 min at 37° C. in a shaking waterbath. The digested cell suspension was passed through a sterile muslin gauze, filtered through 100 μm first, then through 70 μm and the cell-containing filtrate was washed with PBS, as described above. The cell suspension was pelleted by centrifuging for 5 min at 1500 rpm, and the pellet was resuspended in PBS. Cells were layered onto a Percoll gradient (GE Healthcare) (70%-50%-25%) and spun for 30 min without brake at 2,000 rpm. The leukocyte layers at the interfaces between 25% & 50% and between 50% & 70% were collected were collected and washed in PBS.

Blood samples (mother's blood and cord blood), were used as controls for haplotyping and to isolate blood monocytes as a benchmark. Blood monocytes from both, mother and baby, are present in the villi, and the separate preparation of pure monocytes from blood allows the easier identification of these cells in the cell preparation derived from villi. Blood monocytes were processed using the commercial SepMate protocol (STEMCELL Technologies). Briefly, diluted blood was layered onto SepMate tubes and centrifuged at 1200 g for 10 min with break on. The leukocyte ring in the center was collected and washed in PBS by resuspension and gentle centrifugation, as described above.

### Pre-Selection of Cells by AutoMACS

After the leukocyte layers were collected from villous tissue or decidual tissue, the respective cell suspensions were washed with PBS and RBC lysis buffer (Sigma) in order to eliminate contamination by red blood cells. After red blood cell lysis, the cell suspensions (from placental villi or the decidua; all processed separately) were resuspended in MACS buffer (PBS 1X containing 2 mM EDTA and 0.2% FCS). All samples were blocked with 1:100 CD16/32 (2.4G2 BD PharMingen) before surface staining was done on ice with antibodies against EGFR and CD10. The cells which were negative for surface staining of EGFR and CD10 were isolated using AutoMACS (Miltenyi). Since the EGFR+ and CD10+ cells are cyto-trophoblasts and fibroblasts, respectively, selecting EGFR- and CD10-cells at this step eliminated those contaminants, leaving behind a purified cell preparation comprising mostly immune cells.

Leukocytes from cord blood or mother blood were collected separately, as described above, and red blood cell lysis was performed as above to eliminate contamination by red blood cells. Cell suspensions were washed with PBS and processed for further analysis without any pre-selection.

10× Genomics scRNA-seq and data analysis

In order to identify all immune cell populations present in the placental environment by scRNAseq, the cell suspensions were barcoded and pooled.

EGFR− CD10-cell suspensions prepared from decidua or villous tissue, leukocytes from cord blood or mother's blood were separately resuspended in FACS buffer (PBSIX containing 2 mM EDTA and 0.5% FCS) containing CD45 antibody with barcodes. The CD45+ cells within the respective cell suspensions were FACS sorted for 10× Genomics droplet RNA-sequencing to focus on identifying sub-populations of immune cells at the single cell level.

Briefly, an aliquot of the single cell suspension was visually inspected under a light microscope to check viability and cell concentration. As more than 70% of the cells were viable, the cell suspension was diluted according to manufacturer's recommendation to encapsulate about 10,000 cells. The cells were carefully mixed with reverse transcription mix before loading them in a Chromium Single Cell G Chip on the 10× Genomics Chromium system (Zheng et al., 2017) and processed further following the guidelines of the 10× Genomics user manual (v3). In short, the droplets were directly subjected to reverse transcription, the emulsion was broken and cDNA was purified using silane beads. After amplification of cDNA with 12 cycles using primers to enrich cDNA as well as Totalseq-A hashtag, the reaction mix underwent a cleanup, including a fractionation of small fragments (up to 400 bp) to enrich the hashtag sequences and larger fragments (>400 bp) to separate cDNA fragments.

After quality check and quantification, the 10× Genomics single cell RNA-seq library preparation—involving fragmentation, dA-Tailing, adapter ligation and a 10 cycles indexing PCR—was performed based on the manufacturer's protocol. In parallel, the hashtag library was prepared by an 8-cycles index PCR. After quantification, both libraries were sequenced on an Illumina Novaseq 6000 in paired-end mode (R1:29 bp, R2:93 bp), thus generating between ˜11-24 K fragments per cell for the transcript library and ˜5 K fragments per cell for the hashtag library.

To build the reference for Cell Ranger (v3.1.0; provided by 10× Genomics), genome reference (hg38) as well as gene annotation (Ensembl 87) were downloaded from Ensembl. The annotation was filtered with the ‘mkgtf’ command of Cell Ranger (options: ‘--attribute=gene_biotype: protein_coding--attribute=gene_biotype: lincRNA attribute=gene_biotype: antisense’). Genome sequence and filtered annotation were then used as input to the ‘mkref’ command of Cell Ranger to build the appropriate reference. Transcript and hashtag libraries were processed with the ‘count’ command of Cell Ranger. The feature reference for the hashtag library was set up for TotalSeq-A according to Cell Ranger documentation. Cell Ranger was run with option ‘--expect-cells’ set to 10,000 (all other options as per default).

Downstream analysis of each sample was performed using Seurat (v3.0; Tim Stuart, Andrew Butler, Paul Hoffman, Christoph Hafemeister, Efthymia Papalexi, William M. Mauck, Yuhan Hao, Marlon Stoeckius, Peter Smibert, Rahul Satija. Comprehensive Integration of Single-Cell Data, Cell, Volume 177, Issue 7, 2019, Pages 1888-1902.e21. Cells with <200 or >2500 detected genes, and >20% mitochondrial counts were discarded. Genes expressed in <3 cells were discared as well. Gene expressions were then log-normalized and the top 2000 high variable genes were selected by using the “vst” method. The highly variable gene expressions were scaled for principal component analysis (PCA) and further cluster analysis. Clusters were identified using the FindNeighbors ( ) and FindClusters ( ) functions in Seurat. Cell types were annotated by using SingleR (Aran, Looney, Liu et al.: “Reference-based analysis of lung single-cell sequencing reveals a transitional profibrotic macrophage.” Nature Immunology, 20:163-172 (2019)). UMAP and tSNE plots were generated using the RunUMAP ( ) and RuntSNE ( ) function in Seurat. Genes expressed in >25% of cells in clusters were used for differential analysis. Significant differentially expressed genes (DEG) were identified using the FindMarkers ( ) and FindAllMarkers ( ) functions, using the Wilcoxon rank sum test, followed by correcting multiple comparisons.

### Results

### Heterogeneity in Placental Embryonic Macrophages (PEMs):

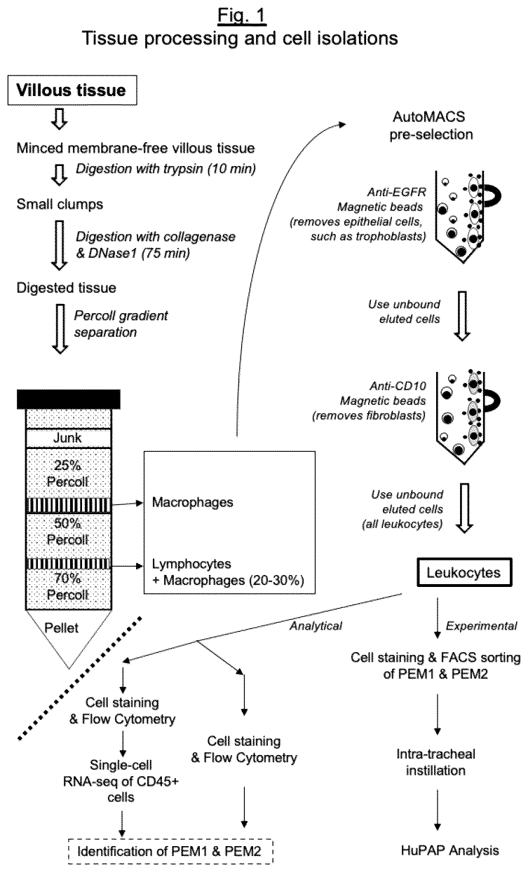

Using multiparametric flow cytometry and transcriptomic analysis, placental macrophages identified as hofbauer cells (HBCs) were shown to be a homogenous population during the first-trimester (Thomas et al., J Exp Med, 2020). HBCs at term or third-trimester have not been well studied. As there are different waves of hematopoiesis during the embryonic developmental stages (Carnegie Stages: CS1 to CS17), we asked whether there is heterogencity in the HBCs or, to use a more general term, in placental embryonic macrophages (PEMs) at term. We first sought to determine this by taking a single cell RNA sequence approach (10x Genomics and Drop seq method), as described above. To do so, and to distinguish between maternal and fetal cells, we took the following tissues for our study: maternal peripheral blood (MB) and decidua basalis of the placenta (DB) as maternal control cells. Fetal cord blood (CB) and villous tissue as fetal derived cells. Placental villous tissue consists of fetal cells inside and maternal cells outside the villous. Thus, this makes it demanding to avoid the contamination with maternal cells during isolation and separation protocols. Based on a previously published isolation protocol (Tang et al., 2011), we adapted our own isolation method, as detailed in the Materials and Methods section above. A schematic overview of the preparation process for placenta-derived leukocytes and the experiments which were performed with the leukocyte-preparation is provided in FIG. 1.

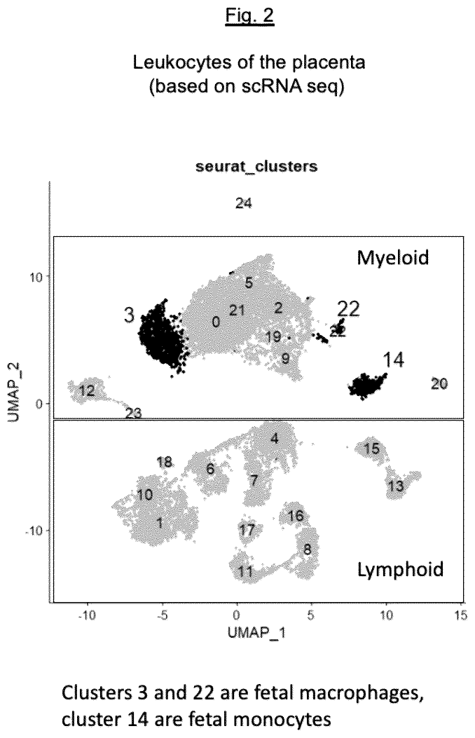

Once cell suspensions from the different tissues mentioned above were isolated, the CD45+immune cells, corresponding to differentiated hematopoietic cells, were sorted and subjected to single cell RNA sequencing, as described in the Materials and Methods section above. Taking into account the tissue origin (villous, decidua, mother blood, cord blood), sex (Maternal: female XX; Fetal: male XY), gene expression pattern and cell type annotation, we could identify lymphoid, crythroid and myeloid clusters in the scRNAseq data. To identify the sub-populations and to check the heterogeneity among the fetal derived macrophages, we focused only on myeloid clusters and re-clustered. We repeated the analysis to verify tissue origin (Villous, Decidua, Mother Blood, Cord Blood), sex typing (Maternal: female XX; Fetal: male XY), gene expression patterns and cell type annotation. Thus, finally we identified three crucial fetal derived myeloid clusters: one cluster of monocytes (cord blood monocytes; cluster 14) and TWO clusters of macrophages (PEM2/cluster 3, and PEM 1/cluster 22) (FIG. 2). We further did DEG (Differentially Expressed Genc) analysis and found that the cluster PEM1 was somewhat related to hofbauer cells which have previously been described to appear during the first-trimester. The cluster PEM2, however, showed macrophage markers at very different gene expression levels when compared to PEM1 and to first trimester HBCs. The list of specific genes or markers for PEM1 and PEM2 in comparison to one another are listed in Table 1.

### First Characterization of Cell Types by Flow Cytometry and FACS

As the next step we asked, whether we could identify the two placental macrophage populations PEM1 and PEM2 within the cell suspension prepared from villous tissue based on surface markers. The EGFR− CD10-cell suspension isolated from villous tissue was resuspended in FACS buffer (PBSIX containing 2 mM EDTA and 0.5% FCS). All samples were subjected to surface staining on ice with antibodies to CD45, CD14, HLA-DR, LYVE1, SIGLEC1 (CD169), MRC1 (CD206), FOLR2, CD55, CD44, CD48, CD89, HLA-A2, HLA-A3, CCR2, CD3, CD19, CD56 and CD66b, and incubated for 30 min at 4C. These antibodies were selected based on surface markers which were predicted to be differently expressed based on the DEG results from the 10× Genomics Single Cell Analysis. Flow cytometry analysis and cell sorting were performed on LSRII Fortessa and FACS Fusion (Becton Dickinson). Cells were gated as follows: Cells were first gated based on cell size and granularity, live cells (eliminating dead cells), singlets (eliminating doublets), and CD45+ (immune cells) cells.

The starting material for the preparation of fetal macrophages from villous tissue also contains some maternal and fetal blood and maternal macrophages. Thus, the cell suspension prepared from villous tissue, while being enriched in fetal macrophages, also contains some contaminants, which we could separate by FACS. Based on haplotyping (HLA-matching) the mother cells (Ex: HLA-A3+) and fetal cells (Ex: HLA-A2+) were separated first. As a next step lymphoid cells were eliminated: T cells based on CD3+, B cells based on CD19+, NK cells based on CD56+, granulocytes based on CD66b+ and blood monocytes based on CCR2+. The resulting negatively gated cells (CD3− CD19− CD56− CD66b− CCR2−) were considered as being Lineage negative (Lin−) cells. Lin− cells were then gated for CD14+ (macrophage) cells. PEM1 and PEM2 appeared as two distinct population, which differed in the strength of their LYVE1 and SIGLEC1 signals.

Further, Lin− CD14+ cells were gated as follows: LYVE1+SIGLEC1+(PEM1) and LYVE1−SIGLEC1−(PEM2). Once these two sub-populations were identified and isolated, the expression of further surface markers—FOLR2, CD206, CD55, CD44, CD48 and CD89-was analyzed.

PEM1 were FOLR2high, CD206⁺, CD55⁺, CD44high, CD48⁺ and CD89⁺.

PEM2 were FOLR2⁺, CD206⁻, CD55high, CD44⁺, CD48⁺ and CD89high

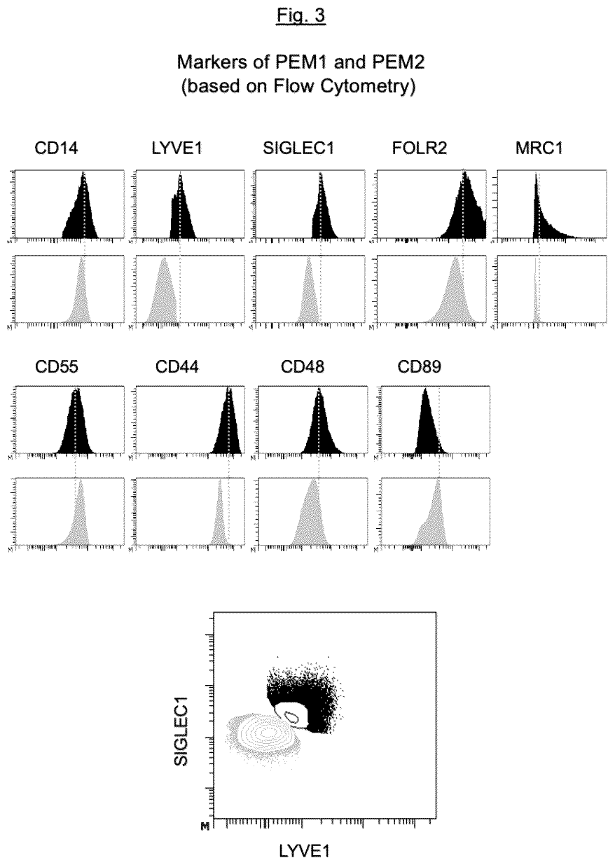

FIG. 3 shows PEM1 macrophages and PEM2 macrophages as two distinct populations, which are clearly separable by their differences in the expression levels of the surface markers Siglec1 and Lyve1.

In a control experiment, and for comparison, we also characterized the other cell types, such as decidual macrophages. Decidual macrophages were gated as follows: the EGFR− CD10-cell suspension was resuspended in FACS buffer (PBSIX containing 2 mM EDTA and 0.5% FCS). All samples were subjected to surface staining on ice with antibodies to CD45, CD14, HLA-DR, HLA-A2, HLA-A3, CCR2, CD3, CD19, CD56 and CD66b, and incubated for 30 min at 4C. Flow cytometry analyses and cell sorting were performed on LSRII Fortessa and FACS Fusion (Becton Dickinson). Cells were gated as follows: cells were first gated based on cell size and granularity, singlets (eliminating doublets), live cells (eliminating dead cells) and CD45+ (immune cells) cells. During Villous tissue preparation steps maternal and fetal blood, maternal decidual cells can get contaminated with fetal immune cells. Based on haplotyping (HLA-matching) mother cells (Ex: HLA-A3+) and fetal cells (Ex: HLA-A2+) were identified first. As a next step lymphoid cells were eliminated, as described above: CD3+ T cells, CD19+B cells, CD56+NK cells, CD66b+Granulocytes and CCR2+blood monocytes. Negatively gated cells (CD3− CD19− CD56− CD66b− CCR2−) were considered as Lineage negative (Lin−) cells. Lin-cells are gated for CD14+HLA−DR+ cells (macrophage) cells.

Blood samples gating (to have a reference for maternal and fetal blood monocytes, respectively):

Mother blood monocytes were Lin−(CD3− CD19− CD56− CD66b−) CCR2+HLA-A3+ CD14+.

Cord blood monocytes were Lin−(CD3− CD19− CD56− CD66b−) CCR2+HLA-A2+ CD14+.

### Further Characterization by FACS and Isolation of PEM1, PEM2 and CB-MO Using Flow Cytometry

For flow cytometry, cells were blocked with FcR Blocking Reagent (Miltenyi, #130-059-901) for 15 minutes at 4° C., washed and stained with antibodies according to Table 2 for 30 minutes at 4° C. Cells were recorded on an LSR Fortessa (BD) or AURORA (Cytek) cytometer and analyzed with FlowJo (BD) or FCS Express 7. Haplo-typing was used to distinguish between maternal and fetal cells. We focused on samples where we can clearly identify different haplo-types on maternal and fetal cells.

Based on the surface staining and analysis we could identify that

PEM1 arc CD45+ CD3− CD19− CD56− CD66b-CCR2-HLA-A2+ CD14+ LYVE1+ SIGLEC1+ FOLR2+ MRC1+;

PEM2 are CD45+ CD3− CD19− CD56− CD66b-CCR2-HLA-A2+ CD14+ LYVE1− SIGLEC1− FOLR2+ MRC1−;

CB-MQ are CD45+ CD3− CD19− CD56− CD66b-CCR2+ HLA-A2+ CD14+

| Target | Company | Cat. Number | Dilution |
|---|---|---|---|
| CD45. BV605 | BioLegend | 304042 | 1:400 |
| CD14. PerCp | BioLegend | 325632 | 1:100 |
| HLA-DR. FITC | BioLegend | 307604 | 1:500 |
| LYVE1. APC | RD systems | FAB20892A | 1:100 |
| SIGLEC1. BUV395 | BD OptiBuild | 751877 | 1:100 |
| FOLR2. PE | BioLegend | 391704 | 1:400 |
| MRC1. PECy7 | Invitrogen | 25-2069-41 | 1:200 |
| HLA-A2. APC-Cy7 | BioLegend | 343310 | 1:300 |
| HLA-A3. BV650 | BD | 747774 | 1:300 |
| CCR2. BV786 | BD OptiBuild | 747855 | 1:300 |
| CD19. PECy5 | BioLegend | 302209 | 1:500 |
| CD3. PECy5 | BD | 555341 | 1:500 |
| CD56. PECy5 | BD | 555517 | 1:500 |
| CD66b. PB | BioLegend | 305111 | 1:500 |
| CD55. FITC | BioLegend | 311306 | 1:100 |
| CD48. BV510 | BD | 563536 | 1:200 |
| CD44. FITC | Invitrogen | 11-0441-82 | 1:100 |
| CD89. PB | BioLegend | 354120 | 1:200 |

We further confirmed these expression data by doing Bulk RNA-seq analysis on isolated PEM1, PEM2 and cord blood monocyte samples. As explained above for the cells derived from villous tissue and for the cord blood cells, the same respective gating strategies were used and 100,000 cells for each sub-population were FACS sorted based on the following respective markers:

PEM1: (CD45+ CD3− CD19− CD56− CD66b− CCR2− HLA-A2+ CD14+ LYVE1+ SIGLEC1+ FOLR2high MRC1+)

PEM2: (CD45+ CD3− CD19− CD56− CD66b− CCR2− HLA-A2+ CD14+ LYVE1-SIGLEC1-FOLR2+ MRC1−)

CB-MQ (CD45+ CD3− CD19− CD56− CD66b-HLA-A2+ CD14+).

For bulk RNA seq, cells were FACS sorted into RLT (Qiagen; a lysis buffer for lysing cells and tissues prior to RNA isolation) buffer containing B-ME. Cells were isolated from three different mothers' (carrying male fetus) samples. RNA was extracted using Qiagen RNeasy Plus Kit (Qiagen Biotech) according to the manufacturers' instructions. RNA quantity was measured by nanodrop and triplicates were submitted for bulk RNA-sequencing.

TABLE 1
We have observed the following gene expression levels in % relative to GAPDH mRNA:

| | IL18RAP | PPBP | S100A12 | SERPINB2 | PTGES | LRG1 | CFP | PTX3 | APOBEC3A | CD55 |
|---|---|---|---|---|---|---|---|---|---|---|
| PEM1 | <0.005 | <0.005 | 0.03-0.3 | 0.1-0.9 | 0.01-0.05 | 0.1-0.5 | 0.2-2.0 | 0.2-0.7 | 0.8-2.0 | 1-5 |
| PEM2 | 0.03-0.20 | 0.01-0.10 | 5-30 | 20-120 | 1.5-6.0 | 10-20 | 12-30 | 4-10 | 11-30 | 15-60 |

| | MRC1 | PLTP | CCL18 | RNASE1 | LGMN | C1QB | ADORA3 | SIGLEC11 | MAF |
|---|---|---|---|---|---|---|---|---|---|
| PEM1 | 5-30 | 12-90 | 1.5-6.0 | 20-150 | 40-90 | 30-180 | 1.5-5.0 | 0.11-0.60 | 2.5-13.0 |
| PEM2 | 0.05-0.5 | 0.5-2.0 | 0.0-0.1 | 0.8-2.0 | 1.5-5.0 | 1.1-4.0 | 0.0-0.09 | 0.0-0.10 | 0.15-0.60 |

Also the following genes show different gene expression patterns between PEM1 and PEM2: SIGLEC1 (high in PEM1, medium high in PEM2); HLA-DOA (h to vh in PEM1, m to h in PEM2); CCL17 (1 in PEM1, <low in PEM2); FOLR2 (h to vh in PEM1, 1 to m in PEM2), LYVE1 (m to vh in PEM1, 1 in PEM2) and FCAR (m in PEM1, vh in PEM2).

### Assessing the Regenerative Potential of PEM1 and PEM2 In Vivo: HuPAP Rescue

PEM1, PEM2 and CB-MO cells were isolated and purified by FACS-sorting into PBS, based on the gating strategies as explained above for the generation of bulk RNA seq data for PEM1 and PEM2. We did two intra-tracheal transplantations of one million cells each which were one week apart from one another for PEM1, PEM2 and CB-MO cells (one million cells in 40 μl PBS per transplantation; total cells per mouse: two million cells) into HuPAP humanized mice, which are an animal model for pulmonary alveolar proteinosis (PAP), a human lung disease (Official name: C;129S4-Rag2tm1.1Flv Csf2/113tm1.1 (CSF2, IL3) Flv Il2rgtm1.1Flv/J; JAX #014595). Mice were analyzed 6 weeks after the first cell transplantation for a) cell engraftment (BAL) using flow cytometry by antibody staining for human CD45+, human CD14+ and mouse CD45-cells;b) BAL fluid ELISA to check human GM-CSF and c) for BAL fluid total protein using BCA kit.

### Human Embryonic Macrophages PEM1 & PEM2 are Functional and Treat Pulmonary Alveolar Proteinosis in a Humanized Mouse Model

To test the in vivo functionality of PEM1 & PEM2 macrophages, we selected a mouse strain called huPAP, which is an animal model for pulmonary alveolar proteinosis (PAP), a human lung disease (Official name: C;129S4-Rag2tm1.1Flv Csf2/113tm1.1 (CSF2, IL3) Flv Il2rgtm1.1Flv/J; JAX #014595). The huPAP strain is an immuno-deficient mouse line designed for transplantation of human cells. Due to the lack of murine GM-CSF expression, the lungs are devoid of alveolar macrophages, hence the mice show signs of alveolar proteinosis, e.g., high protein content in the fluid obtained through bronchoalveolar lavage (BAL), resulting in higher turbidity. Instead of murine GM-CSF, huPAP mice express human GM-CSF (and IL-3), allowing not only the reconstitution of alveolar macrophages with transplanted human cells but also the rescue of the alveolar proteinosis phenotype. Thus, this model is a useful tool to study in vivo functionality of human macrophages.

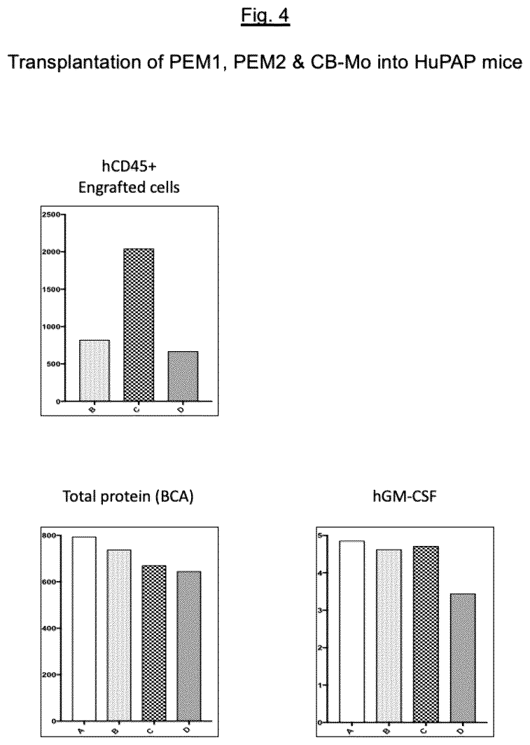

We transplanted equal numbers (1x106 cells per transplantation) of either PEM1, PEM2 or CB-MO macrophages intratracheally into huPAP mice (results in FIG. 4) to check for both cellular engraftment (upper panel) and rescue of PAP. Control mice received an equal volume of PBS (cell resuspension buffer). Human cord blood monocytes were used as a positive control. Typically, four or more cell transplantations of 8 or more millions of human macrophages or monocytes have been used in the published literature to rescue the huPAP phenotype (Happle et al., 2018). Since we were limited by cell availability at the right time for the experiment—the timing of the cesarian sections were not determined by the experiment, but by nature's will-only two transplantations could be done, and even small effects in the huPAP rescue experiment are therefore significant in particular if the rescue effect is as good as or even better than the rescue effect of the positive control (the cord blood monocytes).

HuPAP recipients of both PEM1 & PEM2 macrophages recovered quickly after each transplantation and did not demonstrate abnormal behavior compared to PBS-treated or non-treated animals (data not shown). All animals were transplanted 2 times with PEM1 or PEM2 macrophages, each transplantation separated by 1 week. The animals were analyzed four weeks after the last transplantation.

We found that both PEM1 & PEM2 macrophages showed engraftment into huPAP mice which was as good as that of CB-MO (FIG. 4, upper panel; D=PEM2) or better (FIG. 4, upper panel; C=PEM1) and an improved rescue of the PAP phenotype, demonstrated by a stronger reduction of protein concentration (FIG. 4, lower left panel) and a consumption of human GM-CSF (FIG. 4, lower right panel) in the bronchoalveolar lavage fluid which was as good as (PEM1) or better (PEM2) than the respective rescue observed for the positive control CB-MO. Thus, PEM1 and PEM2 are functional macrophage populations which are suitable for macrophage cell therapy.

### Abbreviations:

HLA: Human Leukocyte Antigen, PBS: Phosphate Buffered Saline, EDTA: Ethylenediaminetetraacetic acid, DMEM: Dulbecco's Modified Eagle's Medium, NCS: Newborn Calf Serum, FCS: Fetal Calf Serum, MACS: Magnetic-activated Cell Sorting, FACS: Fluorescence-activated Cell Sorting, EGFR: Epidermal Growth Factor Receptor, PEM: Placental Embryonic Macrophages, HBC: Hofbauer Cells, RBC: Red Blood Cells. RLT buffer: lysis buffer for lysing cells and tissues prior to RNA isolation.

1. A method of using human term placenta as a source of haplotype-matched placental cells for cell therapy.

2. The method according to claim 1, wherein the placental cells are fetal macrophages.

3. The method according to claim 1, wherein a fetal genotype with regard to HLA-A, HLA-B and HLA-DR is determined prior to birth.

4. The method according to claim 1, wherein a fetal blood type is determined prior to birth.

5. The method according to claim 1, wherein a haplotype-matched human subject in need of cell therapy is identified in a database.

6. Human placental macrophage, the macrophage comprising
presence of surface markers CD45 and CD14; and
presence of surface markers FOLR2 and MRC1, or absence of surface markers MRC1 and SIGLEC1,
for use as a medicament.

7. (canceled)

8. The human placental macrophage according to claim 6, the macrophage further comprising absence of surface markers CD3, CD19, CD56, CD66b and CCR2.

9. The human placental macrophage according to claim 6, wherein the macrophage does not comprise blood-type antigens A and B.

10. The human placental macrophage according to claim 6, wherein the macrophage does not comprise Rhesus D-antigen.

11. The human placental macrophage according to claim 6, wherein the macrophage is homozygous for genes encoding human leukocyte antigens HLA-A, HLA-B or HLA-DR.

12. (canceled)

13. A method for isolation of human placental macrophages from human term placenta, comprising the steps of
a. obtaining villous-containing tissue from the placenta;
b. disintegrating said villous-containing tissue;
c. separating human placental macrophages from debris, cytotrophoblasts and fibroblasts to obtain a cell population comprising placental macrophages; and
d. isolating human placental macrophages by selecting cells comprising the presence of the surface markers CD45, CD14, FOLR2 and MRC1, or cells comprising surface markers CD45 and CD14 and not comprising surface markers MRC1 and SIGLEC1.
